(19) United States
(12) Patent Application Publication
(10) Pub. No.: US 2025/0094688 A1
(43) Pub. Date: Mar. 20, 2025

(54) SUMMARY GENERATION SYSTEM

(71) Applicant: Oracle International Corporation, Redwood Sores, CA (US)

(72) Inventors: Ankit Kumar Aggarwal, Mumbai (IN); Jie Xing, Redmond, WA (US); Haad kahn, Austin, TX (US)

(73) Assignee: Oracle International Corporation, Redwood Shores, CA (US)

(21) Appl. No.: 18/780,384

(22) Filed: Jul. 22, 2024

(30) Foreign Application Priority Data
Sep. 15, 2023 (IN) 202341062232

(51) Int. Cl. G06F 40/166; G06F 40/289; G16H 50/70

(52) U.S. Cl. CPC G06F 40/166; G06F 40/289; G16H 50/70

(57) ABSTRACT

A summary generation system is disclosed that is configured to generate a summary for content to be summarized by identifying relevant chunks of information from the content to be summarized using a large language model (LLM) and a set of questions. The set of questions enable the system to identify and retrieve relevant chunks of information. Each question undergoes a translation or transformation process to generate multiple question variants for each question. The multiple question variants are used by the system to optimize the search to obtain relevant chunks of information. Then, using the multiple question variants and an LLM, the system extracts information (i.e., answers) from the relevant chunks of information. The summary generation system then collates the answers to create an accurate and comprehensive summary for the content to be summarized.

### CROSS-REFERENCES TO RELATED APPLICATIONS

The present application is a non-provisional application of and claims the benefit and priority to India Provisional Application No. 202341062232, filed Sep. 15, 2023, entitled “RETRIEVAL-AUGMENTED GENERATION SYSTEM FOR SUMMARIZING HOSPITAL NOTES INTO COMPREHENSIVE DISCHARGE SUMMARIES,” the entire contents of which are incorporated herein by reference for all purposes.

### BACKGROUND

In today's information-rich age, the volume of data that is generated is extremely large. The success or failure of a user (e.g., a human user, a company, etc.) of that data many times depends on their ability to comprehend the data quickly. In many use cases, given the timeframe available for comprehending the data, it is impossible for the user to read or review all the original data instead the user has to rely on a summary of the data. Summarization is a process that generates a summary for some data, where the length or size of the summary is far less than that of the original data being summarized. A summary is typically a shortened or condensed version of much larger data content that retains the main themes or concepts or ideas described in the larger content. A good summary is one that properly and accurately represents the content being summarized.

In the past, summaries were manually generated. This took a lot of effort and time. With the rise of artificial intelligence techniques, and particularly with the rising popularity of Large Language Models (LLMs), LLMs are used to generate summaries for various types of content, such as documents, webpages, news articles, research papers, etc. This has made it substantially easier to generate summaries very quickly. In certain approaches, prompt engineering techniques have been used to guide the LLMs to retrieve the most relevant information for generating comprehensive and accurate summaries. In prompt engineering, the LLM is provided with a cue or a prompt and the LLM responds to the prompt with relevant information or actions. However, prompt engineering techniques alone are typically not sufficient to generate good summaries. To ensure the capture of relevant information by the LLMs, the LLM can further be fine-tuned using labeled data. The fine-tuning process adapts the LLMs to the summarization task by optimizing its parameters for generating concise and relevant summaries. By fine-tuning a pre-trained LLM using labeled data, the input parameters provided to the model can be customized to generate concise and relevant summaries. These input parameters may include, for instance, the configuration of the model, the prompt provided to the model, the summarization strategy used by the model (e.g., chain of thought prompting technique, a multi-hop question answer evaluation), the temperature setting used for the model to control the randomness of the generated summary and so on.

While fine-tuning techniques can be effective in guiding an LLM to generate concise and relevant summaries, the process often requires hundreds or even thousands of examples (labeled data) to solve the problem of relevance adequately. Securing such a volume of high-quality, labeled data presents its own challenges, both in terms of availability and other security considerations. Furthermore, synthetic data can introduce skewness and biases, potentially causing the LLM to fail in unexpected ways. It is thus a non-trivial and technically challenging task to extract relevant content from content to be summarized in order to generate a summary that can properly and accurately represent the content being summarized.

### BRIEF SUMMARY

The present disclosure relates generally to generating a summary for content to be summarized. More specifically, a summary generation technique is disclosed, where the technique is configured to generate a summary for content to be summarized by identifying relevant chunks of information from the content to be summarized using a large language model (LLM) and a set of questions. The set of questions enable the system to identify and retrieve relevant chunks of information. Each question undergoes a translation or transformation process to generate multiple question variants for each question. The multiple question variants are used by the system to optimize the search to identify relevant chunks of information. Then, using the multiple question variants and an LLM, the technique extracts information (i.e., answers) from the relevant chunks of information. The technique then collates the answers to create an accurate and comprehensive summary for the content to be summarized.

In certain embodiments, a summary generation system is disclosed. The system receives a request to generate a summary for content to be summarized. The system accesses reference information to be used for generating a summary for content to be summarized. The reference information includes a set of original questions and a set of variant questions. The set of variant questions comprises multiple variant questions for each original question in the set of original questions. The system then creates multiple chunks based on the content to be summarized. The system generates an answer for each variant question in the set of variant questions based upon one or more chunks from the multiple chunks and then generates an answer for each original question in the set of original questions based upon the answers generated for the variant questions corresponding to the set of original questions. The system selects a set of answers from the answers generated for the set of original questions and uses a Machine Learning (ML) model and the selected set of answers to generate a summary for the content to be summarized. The system then outputs the summary to a requesting user. As part of generating the summary, in certain examples, the system may display the summary via a UI of a computing device of the requesting user.

In certain examples, the system generates an answer for each variant question in the set of variant questions. The system first uses a first technique to identify a first subset of chunks that are relevant to each variant question. The system then uses a second technique to identify a second subset of chunks that are relevant to each variant question from the first subset of chunks. The system then generates an answer for each variant question based upon the second subset of chunks and a second ML model.

In certain examples, the first technique comprises generating multiple summaries for the multiple chunks using a third ML model. The first technique further comprises generating multiple embeddings for the multiple chunks and generating multiple embeddings for the multiple summaries generated for the multiple chunks. The first technique then uses the multiple embeddings for the multiple chunks and the multiple embeddings for the multiple summaries, and an inverted index to rank the multiple chunks based on relevance of each chunk in the multiple chunks to each variant question. In a certain implementation, the inverted search index is created using a Best Match 25 (BM25) search function. In certain examples, the first subset of chunks are selected by ranking the multiple chunks based on relevance of each chunk in the multiple chunks to the variant question.

In certain examples, the second technique is used to identify a second subset of chunks for each variant question from the first subset of chunks identified for the variant question using a cross encoder model. In a certain implementation, the number of chunks in the second subset of chunks is less than the number of chunks in the first subset of chunks.

In certain examples, generating an answer for each variant question based upon the second subset of chunks comprises concatenating the chunks in the second subset of chunks to generate contextual content for the variant question and using the contextual content and the variant question to make a call to the second ML model to cause the second ML model to generate the answer for the variant question based upon the contextual content.

In certain examples, generating an answer for each original question in the set of original questions based upon the answers generated for the one or more variant questions corresponding to the original questions comprises combining the answers generated for the one more variant questions corresponding to the original question to generate the answer for the original question. In certain examples, selecting a set of answers from the answers generated for the set of original questions comprises filtering one or more answers from the answers generated for the set of original questions using a set of filtering criteria.

### BRIEF DESCRIPTION OF THE DRAWINGS

FIG. 1 depicts an example computing environment of a summary generation system that includes capabilities for automatically generating a summary for content to be summarized by identifying relevant chunks of information from the content to be summarized using a set of questions, according to certain embodiments.

FIG. 2 is a simplified flowchart depicting the processing performed by the subsystems of the summary generation system shown in FIG. 1 for generating a summary for content to be summarized, according to certain embodiments.

FIG. 3 is a flowchart depicting additional details of the processing performed by the subsystems of the summary generation system shown in FIG. 1 for generating a summary for content to be summarized, according to certain embodiments.

FIG. 4 is a simplified flowchart depicting the processing performed by one or more subsystems in the summary generation system shown in FIG. 1 to identify a list of relevant chunks from content to be summarized, according to certain embodiments.

FIG. 5 is a block diagram illustrating one pattern for implementing a cloud infrastructure as a service system, according to at least one embodiment.

FIG. 6 is a block diagram illustrating another pattern for implementing a cloud infrastructure as a service system, according to at least one embodiment.

FIG. 7 is a block diagram illustrating another pattern for implementing a cloud infrastructure as a service system, according to at least one embodiment.

FIG. 8 is a block diagram illustrating another pattern for implementing a cloud infrastructure as a service system, according to at least one embodiment.

FIG. 9 is a block diagram illustrating an example computer system, according to at least one embodiment.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

The present disclosure relates generally to generating a summary for content to be summarized. More specifically, a summary generation technique is disclosed, where the technique is configured to generate a summary for content to be summarized by identifying relevant chunks of information from the content to be summarized using a large language model (LLM) and a set of questions. The set of questions enable the system to identify and retrieve relevant chunks of information. Each question undergoes a translation or transformation process to generate multiple question variants for each question. The multiple question variants are used by the system to optimize the search to identify relevant chunks of information. Then, using the multiple question variants and an LLM, the technique extracts information (i.e., answers) from the relevant chunks of information. The technique then collates the answers to create an accurate and comprehensive summary for the content to be summarized.

As described in the Background section, while the use of LLMs has made it relatively easier to generate summaries for some content, the extraction of relevant content by the LLMs is still a challenge. In certain approaches, Retrieval-Augmented Generation (RAG) techniques may be utilized that leverage the in-context learning and reading comprehension capabilities of LLMs to extract portions of content that contain relevant information. The RAG-based technique utilizes lexical search or semantic search algorithms to understand the meaning of words and phrases in context to extract relevant information from the content. While RAG-based techniques can be used to retrieve relevant information and provide them as context for the LLM, they may suffer from certain drawbacks. These techniques often rely on simplistic matching techniques that may result in the retrieval of irrelevant information. When such information is processed and prepared to augment the generation of a response (for example, a summary), it can produce responses that are misleading, incomplete, or contextually off-target.

The present disclosure addresses several deficiencies of the existing approaches described above. A summary generation system is described that uses a multi-stage solution for converting content to be summarized into concise and comprehensive summaries. In a first stage, the system generates multiple chunks from the content to be summarized. In a second stage, the system identifies and retrieves a list of top “x” chunks from the multiple chunks. The list of top “x” chunks are identified by generating contextual embeddings from the chunks. The embeddings represent high-dimensional numeric vectors that can be used by the system to identify and retrieve relevant chunks of information. The system then re-ranks the list of top “x” chunks to identify a list of top “y” chunks from the list of top “x” chunks using a cross encoder model. The re-ranking of the chunks is performed to ensure that chunks of utmost relevance are identified and retrieved. As part of the processing performed in the second stage, the system additionally utilizes a set of comprehensive questions defined by subject matter experts to further guide the retrieval of relevant chunks of information. Each question undergoes a translation or transformation process to generate multiple question variants to optimize the search to identify relevant chunks of information. In a third stage, using the list of top “y” chunks and a large language model (LLM), the system extracts relevant information (i.e., answers) from the set of questions. In certain examples, the system utilizes the multiple variants for each question to generate answers from the relevant chunks of information. The system then collates the answers to create an accurate and comprehensive summary for the content to be summarized.

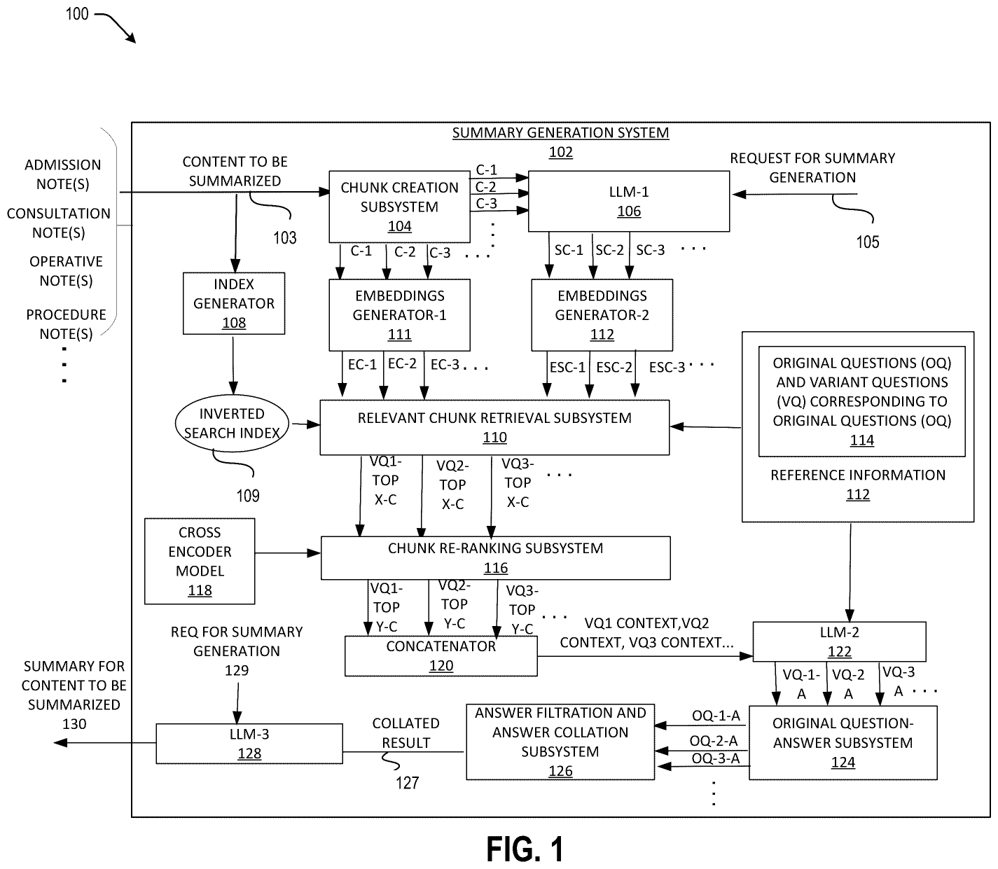

Referring now to the drawings, FIG. 1 depicts an example computing environment 100 of a summary generation system 102 that includes capabilities for automatically generating a summary for content to be summarized by identifying relevant chunks of information from the content to be summarized using a set of questions, according to certain embodiments. The summary generation system 102 may be implemented using one or more computing systems. For example, the computing systems may execute computer-readable instructions (e.g., code, program) to implement the summary generation system 102. As depicted in FIG. 1, the summary generation system 102 includes various computing systems including a chunk creation subsystem 104, a relevant chunk retrieval subsystem 110, a chunk re-ranking subsystem 116, an original question-answer subsystem 124 and an answer filtration and answer collation subsystem 126. The systems and subsystems depicted in FIG. 1 may be implemented using only software (e.g., code, instructions, program) executed by one or more processing units (e.g., processors, cores) of a computing system, hardware, or combinations thereof. The software may be stored on a non-transitory storage medium (e.g., on a memory device).

The summary generation system 102 may be implemented using various different configurations. In certain embodiments, the summary generation system 102 may represent a computing system of an entity (for e.g., an organization, an enterprise, or an individual) that provides summary generation functionality to its users. In other embodiments, the summary generation system 102 may be implemented on one or more servers of a cloud provider network and its summary generation services may be provided to subscribers of cloud services on a subscription basis. The functionality to provide summary generation, as described in this disclosure, may be offered as part of the service. A customer can subscribe to the service to generate a summary for content to be summarized and provide the summary for the content to be summarized to its users. As part of generating the summary, in certain examples, the service may also display the summary via a UI of a computing device of the requesting subscriber as described in this disclosure.

Computing environment 100 depicted in FIG. 1 is merely an example and is not intended to unduly limit the scope of claimed embodiments. One of ordinary skill in the art would recognize many possible variations, alternatives, and modifications. For example, in some implementations, the system can be implemented using more or fewer subsystems than those shown in FIG. 1, may combine two or more subsystems, or may have a different configuration or arrangement of subsystems.

The summary generation system 102 may be configured to receive the content 103 to be summarized in various ways. For instance, in one approach, the summary generation system 102 may receive the content to be summarized via an application of a user device. A user may interact with the system 102 using a user device that is communicatively coupled to the system 102, possibly via one or more communication networks. The user device may be of various types, including but not limited to, a mobile phone, a tablet, a desktop computer, and the like. The user may use the application to input the content to be summarized by the system 102. In other approaches, the content to be summarized may be provided via a cloud service or a third party system to the summary generation system 102.

The summary generation system 102 may be configured to generate a summary for various types of content, including, but not limited to, documents, webpages, news articles, research papers, hospital notes and so on. In certain examples, the content 103 to be summarized comprises a single document. In other examples, the content to be summarized can comprise multiple documents. For instance, in the example depicted in FIG. 1, the content 103 to be summarized comprises multiple documents such as admission notes, consultation notes, operative notes, and progress notes that are related to a patient's stay at a hospital. These documents describe the patient's symptoms, the patient's ailment, the treatment provided to the patient, and the patient's progress during the patient's stay at the hospital. The summary generation system 102 receives the content 103 to be summarized, processes the content, and based on the processing, generates a summary that accurately and properly describes the content being summarized.

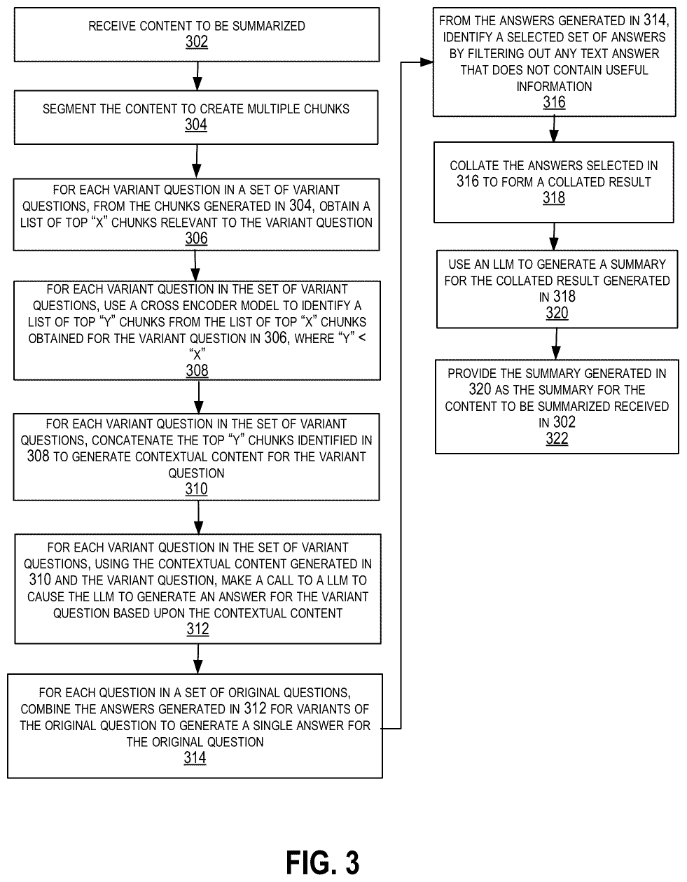

The processing performed by the summary generation system 102 to generate a summary for content to be summarized comprises multiple stages. In a first stage, upon receiving the content 103 to be summarized, the summary generation system 102 segments the content to generate multiple chunks. In a certain implementation, the content 103 to be summarized is received by a chunk creation subsystem 104 within the summary generation system 102. As part of the processing in the first stage, the chunk creation subsystem 104 divides the set of documents (e.g., admission notes, consultation notes, operative notes, and progress notes) representing the content 103 to be summarized into smaller and more manageable chunks. The chunk creation subsystem 104 may utilize various types of techniques to create multiple chunks from the content to be summarized. In a certain implementation, the chunk creation subsystem 104 may utilize a recursive text splitter to create multiple chunks of a particular chunk size (e.g., a paragraph). For instance, in certain examples, the recursive text splitter may divide the documents representing the content to be summarized into multiple paragraphs, where each paragraph is of a certain chunk size (e.g., 500 tokens). If a paragraph is longer than 500 tokens, the text splitter may further break the paragraph into multiple chunks such that there is a chunk overlap of two sentences between two consecutive chunks. Examples of chunks created by the chunk creation subsystem 104 for a set of documents related to a patient's stay at a hospital are depicted in FIG. 3 below.

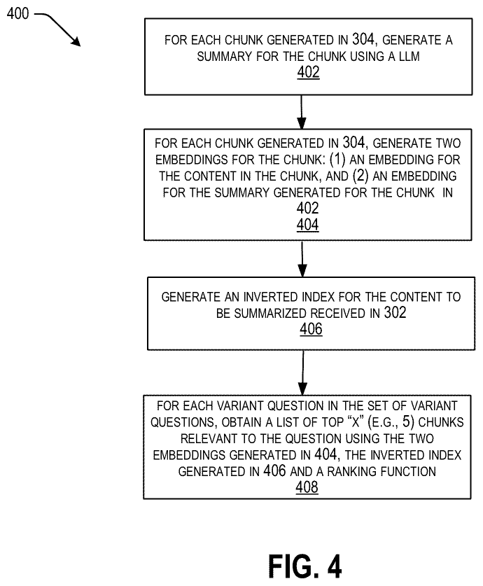

In a second stage of processing, the summary generation system 102 identifies and retrieves a first subset of chunks from the multiple chunks generated by the chunk creation subsystem 104. The first subset of chunks are identified and retrieved by the summary generation system 102 using a set of contextual embeddings generated for the multiple chunks and a set of comprehensive questions defined as part of reference information 112. The set of questions are used to further guide the system to identify and retrieve the first subset of chunks. The first subset of chunks represent a list of top “x” chunks that are identified from the multiple chunks. The list of top “x” chunks are identified by ranking the multiple chunks based on determining the relevance of each chunk in the multiple chunks using the contextual embeddings. To ensure that chunks of utmost relevance are identified and retrieved, the summary generation system 102 then identifies a second subset of relevant chunks based on the first subset of chunks. Details of the processing performed by the summary generation system 102 to identify a first subset of chunks and a second subset of chunks is described in FIG. 3 and FIG. 4.

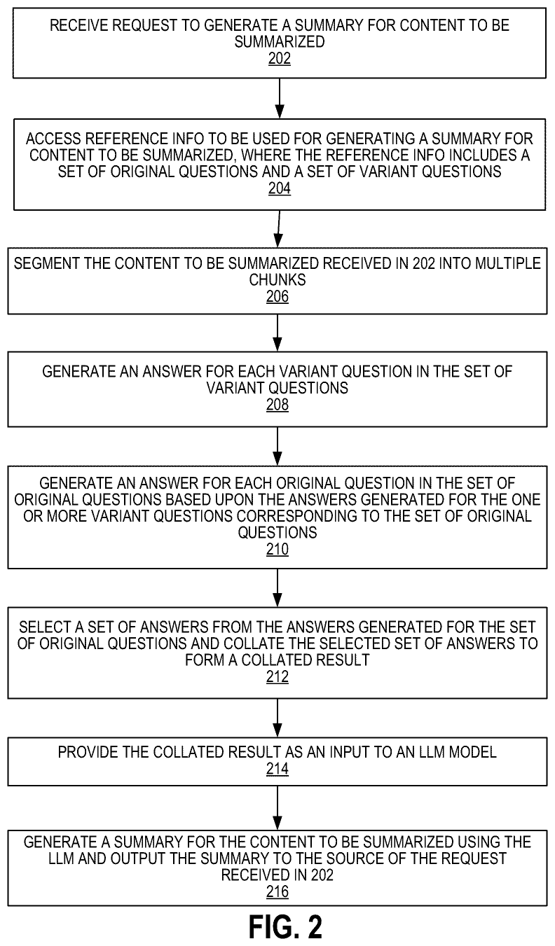

In a third stage of processing, the summary generation system 102 uses the set of questions and a large language model (LLM) to extract relevant information (i.e., answers) from the content of each chunk in the second subset of chunks. The summary generation system 102 then collates the answers and uses a collated result (answer) 127 to create an accurate and comprehensive summary for the content to be summarized. The results of the processing performed by the generation system 102 are then communicated back to the requesting user. These results may include a summary 130 for the content to be summarized. For instance, if the content to be summarized 103 represents a set of documents related to a patient as described above, the results generated by the system may include a hospital discharge summary generated for the patient. Details related to the processing performed by the various subsystems in FIG. 1 for generating a summary for content to be summarized are described below with respect to the flowcharts depicted in FIGS. 2-4 and their accompanying description.

FIG. 2 is a simplified flowchart 200 depicting the processing performed by the subsystems of the summary generation system 102 shown in FIG. 1 for generating a summary for content to be summarized, according to certain embodiments. The processing depicted in FIG. 2 may be implemented in software (e.g., code, instructions, program) executed by one or more processing units (e.g., processors, cores) of the respective systems, using hardware, or combinations thereof. The software may be stored on a non-transitory storage medium (e.g., on a memory device). The method presented in FIG. 2 and described below is intended to be illustrative and non-limiting. Although FIG. 2 depicts the various processing steps occurring in a particular sequence or order, this is not intended to be limiting. In certain alternative embodiments, the processing may be performed in some different order, or some steps may also be performed in parallel. In certain embodiments, such as in the embodiment depicted in FIG. 1, the processing depicted in FIG. 2 may be performed by the subsystems (104, 110, 111, 112, 116, 120, 124, and 126) described in FIG. 1.

In the embodiment depicted in FIG. 2, processing may be initiated at block 202 when the summary generation system receives a request to generate a summary for content (e.g., 103) to be summarized. The content to be summarized may comprise a single document or multiple documents. For instance, in the example described in FIG. 1, the content to be summarized comprises multiple documents such as admission notes, consultation notes, operative notes, and progress notes related to a patient.

At block 204, the summary generation system accesses reference information (e.g., the reference information 112 shown in FIG. 1) to be used for generating a summary for content to be summarized. As described above, the reference information 112 comprises a set of comprehensive questions used by the summary generation system 102 to guide the system to retrieve relevant chunks of information to generate a summary for the content to be summarized. The set of questions may be drafted and selected by Subject Matter Experts (SMEs) based on the domain of the content for which summarization is being performed. For instance, in the context of a medical domain, the reference information 112 may comprise a set of questions that relate to a patient's admission date, the discharge date, personal demographics, clinical data, vitals of the patient, home medications and so on.

In certain embodiments, each question in the set of questions (also referred to herein as a set of original questions) defined in the reference information 112 is transformed to generate multiple variants for each question. For instance, the transformation may include re-phrasing the original question by changing the order of certain words or adding or deleting certain words in the original question while still retaining the meaning of the original question. As will be described in greater detail below, the multiple variants for each question are used by the summary generation system 102 to identify and obtain relevant chunks of information from the content to be summarized. The multiple variants for each question are additionally used by the summary generation system 102 to generate answers from the relevant chunks of information and these answers are utilized by the summary generation system 102 to create an accurate and comprehensive summary for the content to be summarized. An example of a set of original questions and a set of variant questions that correspond to each original question in the set of original questions in the context of a medical domain is shown in Table-1 below.

TABLE 1

| Question | Variant-1 | Variant-2 |
|---|---|---|
| Has the date of admission | Is the admission date | Is there a date he |
| been specified? | given? | came into the |
| | | hospital? |
| Has the date of discharge | Is the discharge date | Is there a date he |
| been mentioned? | mentioned? | left? |
| Are the personal | Do we have the | Do we know his |
| demographics such as | patient's name, age, | name and age? |
| name, age and gender of | and gender? | |
| the patient provided? | | |
| What is the reason for the | Why was the patient | Why did he come |
| patient's admission? | admitted? | to the hospital? |
| Is there a summary of the | Do we have a | Why was he |
| admission criteria or | summary of why they | allowed in? |
| history? | were admitted? | |
| Was a physical exam | Was there a physical | Did a doctor check |
| conducted upon | exam when they | him when he came? |
| admission? If so, is it | came in? Is it written | Is it written down? |
| documented? | down? | |
| Are the clinical data and | Do we have their | Do we have his |
| vitals at the time of | health data and vital | health details from |
| admission provided? | signs from when they | when he came? |
| | arrived? | |
| Is there a summary of the | Do we have a | Do we know about |
| patient's medical and | summary of their past | his past health |
| surgical history? | medical and surgical | problems? |
| | details? | |
| Is there a summary of the | Do we have a list of | Do we know his |
| patient's home medications | their home | usual medicines? |
| and treatments? | medications and | |
| | treatments? | |
| Is the initial treatment | Do we have a | What did the |
| plan for the patient | summary of their first | doctors plan to do |
| summarized? | treatment plan? | for him? |
| Are any operations or | Do we have a | Do we know about |
| procedures the patient | summary of any | any surgeries or |
| underwent summarized? | surgeries or | treatments he had |
| | procedures they had? | here? |
| Is there a periodic | Do we have regular | Are there short |
| summary of events from | summaries from | notes from each |
| the daily notes? | daily notes? | day for him? |
| Are daily progress notes | Are daily updates on | Are there daily |
| for the patient | the patient | updates about him? |
| summarized? | summarized? | |
| Are relevant labs, imaging, | Are tests, pictures, | Did he have any |
| and treatments | and treatments | tests or scans? Are |
| documented? | written down? | they written down? |
| Are there notes on relevant | Are there notes on | Did anything |
| physical findings during | any physical changes | change with his |
| the hospital stay? | during their stay? | body during the |
| | | stay? |
| Were there any | Were there any | Did other experts |
| consultations? If so, are | expert opinions? Are | see him? Is it |
| they summarized? | they summarized? | written down? |
| Is there an assessment of | Do we know if the | Is he ready to |
| the patient's readiness for | patient is ready to | leave? |
| discharge? | leave? | |
| What are the discharge | What are the reasons | Why is he leaving? |
| diagnoses for the patient? | they're being | |
| | discharged? | |
| Is there a plan for the | What's the plan when | How will he be |
| patient's discharge? | the patient leaves? | cared for when |
| | | leaving? |
| What is the disposition | How is the patient | How is he feeling |
| upon discharge? | doing when they | when leaving? |
| | leave? | |
| Is there a follow-up plan | What's the plan after | What should he do |
| post-discharge? | they leave? | after leaving? |
| Are there any special | Are there any special | Should he do |
| activity instructions for the | things the patient | anything special |
| patient post-discharge? | should do after | after leaving? |
| | leaving? | |
| Are there specific dietary | Are there any food | What should he eat |
| recommendations for the | suggestions for the | when leaving? |
| patient upon discharge? | patient when they | |
| | leave? | |
| Are the medications to be | Is there a list of | What medicines |
| taken post-discharge | medications for after | should he take after |
| listed? | they leave? | leaving? |
| Is there a pain | Do we have a plan | How will his pain |
| management plan for the | for managing their | be managed after |
| patient post-discharge? | pain after they leave? | leaving? |
| What is the total length of | How long was the | How many days |
| the patient's stay in the | patient in the | was he in the |
| hospital? | hospital? | hospital? |

Table-1 shown above is merely an example and not intended to unduly limit the scope of claimed embodiments. One of ordinary skill in the art would recognize many possible variations, alternatives, and modifications. For example, in some implementations, Table-1 may be implemented using more or fewer columns, may combine two or more columns of information, or may have different columns than shown in the illustration. Additionally, while the implementation shown in Table-1 depicts certain examples of original questions and their corresponding variant questions, in other implementations, Table-1 may include more or fewer or different examples of original questions and variant questions. For example, the specific implementation shown in Table-1 illustrates two variant questions that are identified for each original question. In other examples, more or fewer variant questions can be identified for each original question.

At block 206, the summary generation system 102 segments the content to be summarized into multiple chunks. As described in FIG. 1, in one implementation, the multiple chunks are created by the chunk creation subsystem 104 in the summary generation system 102. Examples of chunks created by the chunk creation subsystem 104 based on content to be summarized are depicted in FIG. 4 below.

At block 208, the summary generation system 102 generates an answer for each variant question in the set of variant questions (defined as part of the reference information 112) based on a set of relevant chunks that are identified from the multiple chunks created in 206. To identify the set of relevant chunks, the summary generation system 102 first identifies a first subset of chunks (i.e., a list of top “x” chunks) that are relevant to (i.e., answered by) each variant question in the set of variant questions. Additional details of the processing performed by the summary generation system 102 to identify and retrieve a first subset of chunks is described in FIG. 4 below.

Upon identifying and retrieving the first subset of chunks (i.e., a ranked list of top “x” chunks) that are relevant to each variant question, to further improve the accuracy of the relevant information that is extracted for summarization and to ensure that chunks of utmost relevance are identified and retrieved, the summary generation system 102 re-ranks the list of top “x” chunks to identify and obtain a list of top “y” chunks (i.e., a second subset of chunks) that are relevant to each variant question in the set of variant questions. In certain embodiments, the number of chunks in the list of top “y” chunks is less than the number of chunks in the list of top “x” chunks. The second subset of chunks are identified by the system using a cross encoder model. Additional details of the processing performed by the summary generation system 102 to identify and retrieve the second subset of chunks is described in FIG. 3 below.

The summary generation system 102 then generates an answer for each variant question in the set of variant questions based upon the second subset of chunks and a ML model. To generate an answer for each variant question, in one embodiment, and as depicted in FIG. 1, the summary generation system 102 provides the list of top y″ chunks (VQ1-Top Y-C, VQ2-Top Y-C, VQ3-Top Y-C . . . ) to a concatenator 120 which concatenates the chunks in the second subset of chunks (i.e., list of top “y” chunks) generate contextual content for answering each variant question in the set of variant questions. The concatenator 120 then provides the concatenated context (VQ1 context, VQ2 context, VQ3 context . . . ) for answering each variant question in the set of variant questions to a ML model (e.g., LLM-2 122) for further processing. The concatenator 120 makes a call to the LLM-2 122 to cause the model to generate an answer for each variant question based upon the contextual context.

For instance, as part of the processing performed in 208, the LLM-2 122 processes each variant question corresponding to an original question identified in the reference set of questions defined in table-1 to generate an answer for the variant question based upon the contextual context. For instance, an original question and its variants shown in table-1 is illustrated below:
Original Question: “What is the reason for the patient's admission.”
Variant Question 1: Why was the patient admitted.”
Variant Question 2: “Why did he come to the hospital.”

As part of the processing performed in 208, the LLM-2 122 processes each variant question corresponding to the original question to generate an answer for the variant question based upon the contextual context as follows:
Answer 1: The reason for the patient's admission was diverticular bleeding.
Answer 2: The patient is admitted for a lower GI bleed.

At block 210 the summary generation system 102 generates an answer for each original question in the set of original questions based upon the answers generated for one or more variant questions corresponding to the original questions. For instance, in the embodiment depicted in FIG. 1, the set of answers (VQ-1 A, VQ-2 A, VQ-3 A . . . ) generated for each variant question in the set of variant questions are then provided to an original question answer subsystem 124 within the summary generation system 102 for further processing. The original question answer subsystem 124 generates an answer (OQ1-A, OQ2-A, OQ3-A . . . ) for each original question in the set of original questions by combining the answers generated for each variant question corresponding to the original question.

At block 212, the summary generation system 102 selects a set of answers from the answers generated for the set of original questions and collates the selected set of answers to form a collated result. For instance, in the embodiment depicted in FIG. 1, the answer filtration and answer collation subsystem 126 may select a set of answers from the set of answers (OQ1-A, OQ2-A, OQ3-A . . . ) generated for the set of original questions by applying filtering criteria to the generated answers. Various types of filtering criteria can be applied by the answer filtration and answer collation subsystem 126 to select a set of answers from the answers generated for the set of original questions. For instance, the answer filtration and answer collation subsystem 126 may apply filtering criteria that can identify and filter duplicate or irrelevant information from the answers, identify and filter sensitive information from the generated answers and so on.

At block 214, the summary generation system 102 provides the collated result 127 as an input to a LLM model. For instance, in the embodiment depicted in FIG. 1, the answer filtration and answer collation subsystem 126 provides the collated result 127 as an input to a LLM (LLM-3 128).

At block 216, the summary generation system generates a summary for the content to be summarized using the LLM and outputs the summary to the source (e.g., a requesting user) of the request received in 202. An example of a hospital discharge summary that is generated and output by the summary generation system for content to be summarized, where the content represents a set of documents that are related to a patient's stay at a hospital is shown below:
Patient John Doe, a 62-year-old male with a history of hypertension and hyperlipidemia, was admitted for evaluation of lower GI bleed due to diverticulosis in the sigmoid colon. Upon admission, he appeared comfortable with stable vital signs. Initial treatment included IV hydration, packed RBCs, and a gastroenterology consult for a colonoscopy. Throughout his stay, he received appropriate care, including monitoring of hemoglobin levels and management of hypertension and hyperlipidemia. The patients condition improved, with no further bleeding episodes and toleration of a high fiber diet. He will be discharged the following day with instructions to follow up with a gastroenterologist, continue Lisinopril and Atorvastatin, and adhere to a high fiber diet. Pain management post-discharge will include acetaminophen as needed. The patients stable condition and well-being support the decision for discharge after a three-day hospital stay.

### EXAMPLE OF A HOSPITAL DISCHARGE SUMMARY

The hospital discharge summary illustrated above is merely an example of a summary generated by the system for a hospital note and is not intended to unduly limit the scope of claimed embodiments. One of ordinary skill in the art would recognize many possible variations, alternatives, and modifications. For example, in some implementations, the system may generate other different summaries for the same content based upon changes to the configuration of the LLM, the types of prompts provided to the LLM, the summarization strategy used (e.g., chain of thought prompting technique, a multi-hop question answer evaluation) by the LLM, the input parameters (setup) for the model, the temperature setting used for the model to control the randomness, and the like. In addition, while the example illustrated above is an example of a hospital discharge summary that is generated by the system, in alternate embodiments, the system may include capabilities to generate summaries for various other types of content such as such as documents, webpages, news articles, research papers and so on.

FIG. 3 is a flowchart 300 depicting additional details of the processing performed by the subsystems of the summary generation system 102 shown in FIG. 1 for generating a summary for content to be summarized, according to certain embodiments. The processing depicted in FIG. 3 may be implemented in software (e.g., code, instructions, program) executed by one or more processing units (e.g., processors, cores) of the respective systems, using hardware, or combinations thereof. The software may be stored on a non-transitory storage medium (e.g., on a memory device). The method presented in FIG. 3 and described below is intended to be illustrative and non-limiting. Although FIG. 3 depicts the various processing steps occurring in a particular sequence or order, this is not intended to be limiting. In certain alternative embodiments, the processing may be performed in some different order, or some steps may also be performed in parallel. In certain embodiments, such as in the embodiment depicted in FIG. 1, the processing depicted in FIG. 3 may be performed by the subsystems (104, 110, 111, 112, 116, 120, 124, and 126) described in FIG. 1.

In the embodiment depicted in FIG. 3, processing may be initiated at block 302 when the chunk creation subsystem 104 receives content (e.g., 103) to be summarized. As described above, in certain embodiments, the content to be summarized may comprise various types of documents such as admission notes, consultation notes, operative notes, and procedure notes related to patient's stay at the hospital. Example 1 shown below represents an admission note related to a patient, example 2 represents a consultation note related to the patient, example 3 represents an operative note related to the patient and example 4 represents a procedure note related to the patient. The examples shown below are merely illustrations of different types of documents (i.e., content) that can be processed and summarized by the summary generation system. One of ordinary skill in the art would recognize many possible variations, alternatives, and modifications. For example, in some implementations, the summary generation system may be configured to process various other types of content such as webpages, news articles, research papers and so on.

### Example 1 (Admission Note)

History and Physical Note, 6 Jul. 2023 Patient: John Doe, DOB Jan. 14, 1961, MR 6128394
HPI: John Doe is a 62-year-old male with a past medical history of hypertension and hyperlipidemia who presents with a 24-hour history of rectal bleeding. He reports that the bleeding started suddenly yesterday evening, describing the blood as bright red and mixed with stool. He also reports experiencing three episodes of such bleeding, each associated with bowel movements. The amount of blood is significant, enough to turn the toilet water red. He denies any associated abdominal pain, nausea, vomiting, or changes in bowel habits. The patient has never experienced anything similar in the past. He denies any history of peptic ulcer disease, gastritis, or gastrointestinal malignancies. He also denies any recent changes in his diet, alcohol use, or NSAID use.
The patient sought care when he noticed a drop in his energy level. He reported feeling light-headed and slightly short of breath during minimal exertion. He denies chest pain, palpitations, or syncope. He also denies fever, night sweats, weight loss, or other systemic symptoms.
The patient's past medical history is significant for hypertension, which is well controlled with Lisinopril, and hyperlipidemia, managed with Atorvastatin. He reports medication compliance and has not made any recent changes to his medication regimen. He denies tobacco use and uses alcohol occasionally, in moderation. There is no known family history of colorectal cancer or inflammatory bowel disease. He has never had a colonoscopy.
Upon arrival to the emergency department, vital signs showed blood pressure of 110/70 mmHg, heart rate of 100 beats per minute, respiratory rate of 16 breaths per minute, body temperature of 98.6° F., and oxygen saturation of 97% on room air. Physical examination revealed pale conjunctiva and mild tachycardia but was otherwise unremarkable, including a benign abdominal exam. Initial lab tests showed a drop in hemoglobin from his baseline of 14 g/dL to 9 g/dL. Liver function tests, including coagulation parameters, were within normal limits. Given his presentation and these findings, he was admitted to the hospital for further evaluation and management of a lower GI bleed.
Past Medical History: ⋅Hypertension ⋅Hyperlipidemia
Past Surgical History: ⋅Appendectomy in 1983
Social History: Mr. Doe is a retired engineer, married, with 2 grown-up children. He is a lifelong non-smoker and denies illicit drug use. He reports social alcohol use, with an average of 1-2 drinks per week.
Home Medications: ⋅Lisinopril 10 mg daily ⋅Atorvastatin 20 mg daily
Physical Exam: ⋅General—Patient appears comfortable, in no acute distress ⋅HEENT—Normocephalic atraumatic, mucus membranes moist ⋅Cardiovascular—RRR, no murmurs ⋅Respiratory—Clear to auscultation bilaterally ⋅Abdomen—Soft, non-tender, non-distended, no palpable masses or hepatosplenomegaly ⋅Rectal—Bright red blood on glove, no palpable masses, normal sphincter tone ⋅Extremities—Warm and well perfused, no edema.
Labs: ⋅CBC: WBC 8.2, Hgb 11.0, Plt 210 ⋅BMP: Na 139, K 4.0, Cl 104, CO2 26, BUN 18, Cr 0.9, Glucose 102 ⋅Coagulation Profile: PT 11.5, INR 1.1, aPTT 30
Assessment & Plan:
1. Lower GI Bleed: Start IV hydration, transfuse 2 units of packed RBCs, arrange for urgent gastroenterology consult for colonoscopy. Maintain NPO status.
2. Hypertension: Continue with Lisinopril.
3. Hyperlipidemia: Continue with Atorvastatin.
A gastroenterology consult will be obtained for further management.

### Example 2 (Consultation Note)

Gastroenterology Consultation Note, 7 Jul. 2023 Patient: John Doe, DOB Jan. 14, 1961, MR 6128394
Reason for consult: Lower GI Bleed
History of Present Illness: Mr. Doe is a 62-year-old male with a history of hypertension and hyperlipidemia, presented with the complaint of bright red blood per rectum. He reports the symptoms started two days ago with intermittent lower abdominal cramping. There's no known history of GI bleeds, peptic ulcer disease, or liver disease. He denies any recent NSAID or anticoagulant use.
Physical Exam: ⋅General: No acute distress ⋅Cardiovascular: Regular rhythm, no murmurs ⋅Respiratory: Clear to auscultation bilaterally ⋅Abdomen: Soft, non-tender ⋅Rectal: Bright red blood on glove
Laboratory data: ⋅Hgb: 11.0 g/dL (lower from baseline of 14 g/dL) ⋅Coagulation Profile: PT 11.5, INR 1.1, aPTT 30
Assessment:
1. Lower GI bleed: Mr. Doe's presentation of bright red blood per rectum, coupled with a drop in hemoglobin, is consistent with a lower gastrointestinal bleed. The absence of any significant upper GI symptoms and normal liver function tests rules out an upper GI source.
Plan:
1. Colonoscopy: Will schedule an urgent colonoscopy for further evaluation and to identify the source of bleeding.
2. Resuscitation: Continue IV fluids and transfusion of packed red blood cells.
3. GI prophylaxis: Start proton pump inhibitor for gastric mucosa protection.
4. Recheck Labs: Monitor hemoglobin and vital signs closely.

### Example 3 (Operative Note)

Operative Note, 7 Jul. 2023 Patient: John Doe, DOB 01/14/1961, MR 6128394
Procedure: Colonoscopy
Indication: Evaluation of lower GI bleed
Findings: Colonoscopy showed diverticulosis in the sigmoid colon with evidence of recent bleeding.
Procedure: Colonoscopy was performed under conscious sedation. The scope was advanced to the cecum. The colon was carefully examined on withdrawal. There was evidence of diverticulosis in the sigmoid colon. A large clot was noted in one of the diverticula, but no active bleeding was identified.
Impression:
1. Diverticulosis in the sigmoid colon with evidence of recent bleeding
Plan:
1. Diverticular bleeding: The patient should follow a high fiber diet to prevent further episodes of diverticular bleeding.
2. Continue IV fluids and monitor hemoglobin closely.
3. Plan for discharge once stable.

### Example 4 (Progress Note)

Progress Note, 8 Jul. 2023 Patient: John Doe, DOB 01/14/1961, MR 6128394
S: The patient reports no further episodes of rectal bleeding. Denies abdominal pain.
O: ⋅BP 130/75, HR 80, Temp 98.6° F., RR 16, SpO2 98% Physical Exam: ⋅General: Appears comfortable ⋅Cardiovascular: Regular rhythm, no murmurs ⋅Respiratory: Clear to auscultation bilaterally ⋅Abdome: Soft, non-tender ⋅Rectal: No blood on glove Labs: ⋅Hgb: 10.6 g/dL
A:
1. Diverticular Bleeding: Stable, no further bleeding episodes
2. Hypertension: Controlled
3. Hyperlipidemia: Controlled
P:
1. Continue with IV hydration.
2. Monitor hemoglobin.
3. Re-introduce solid food as tolerated.
Progress Note, 9 Jul. 2023 Patient: John Doe, DOB Jan. 14, 1961, MR 6128394
S: The patient reports feeling well, tolerating diet.
O: ⋅BP 125/80, HR 78, Temp 98.4° F., RR 16, SpO2 98% Physical Exam: ⋅General: No acute distress Cardiovascular: Regular rhythm, no murmurs ⋅Respiratory: Clear to auscultation bilaterally. Abdome: Soft, non-tender ⋅Rectal: No blood on glove Labs: ⋅Hgb: 11.0 g/dL
A:
1. Diverticular Bleeding: Stable, no further bleeding episodes
2. Hypertension: Controlled
3. Hyperlipidemia: Controlled
P:
1. Discontinue IV fluids.
2. Continue to monitor hemoglobin.
3. Plan for discharge tomorrow if remains stable.
Progress Note, 10 Jul. 2023 Patient: John Doe, DOB Jan. 14, 1961, MR 6128394
S: The patient reports feeling well, no episodes of rectal bleeding. Tolerating diet.
O: ⋅BP 120/78, HR 77, Temp 98.2° F., RR 16, SpO2 98% Physical Exam: ⋅General: No acute distress Cardiovascular: Regular rhythm, no murmurs ⋅Respiratory: Clear to auscultation bilaterally. Abdome: Soft, non-tender ⋅Rectal: No blood on glove Labs: ⋅Hgb: 11.2 g/dL
A:
1. Diverticular Bleeding: Stable, no further bleeding episodes
2. Hypertension: Controlled
3. Hyperlipidemia: Controlled
P:
1. Patient is stable for discharge.
2. Recommend follow-up with a gastroenterologist in 1-2 weeks.
3. Recommend a high fiber diet to help prevent further episodes of diverticular bleeding.
4. Continue home medications.
5. Patient to return to ER if bleeding recurs or if he experiences any severe abdominal pain, fever, or other new symptoms.

At block 304, the chunk creation subsystem 104 segments the content to create multiple chunks. As described in FIG. 1, in one implementation, the chunk creation subsystem 104 utilizes a recursive text splitter to create multiple chunks of a particular chunk size (e.g., a paragraph). For instance, in certain examples, the recursive text splitter may divide the documents representing the content to be summarized into multiple paragraphs, where each paragraph is of a certain chunk size (e.g., 500 tokens). Examples of chunks created by the subsystem 104 based on content to be summarized are illustrated in FIG. 4 below.

At block 306, the chunk retrieval subsystem identifies and obtains a list of top “x” chunks that are relevant to (i.e., that are answered by) each variant question in the set of variant questions defined in reference information 112. Additional details of the processing performed by the chunk retrieval subsystem to identify and obtain a list of top “x” chunks are described in detail in FIG. 4.

Upon identifying and retrieving a list of top “x” chunks that are relevant to each variant question in the set of variant questions, to further improve the accuracy of the relevant information that is extracted for summarization and to ensure that chunks of utmost relevance are identified and retrieved, the chunk re-ranking subsystem 116 re-ranks the list of top “x” chunks obtained in block 306 to identify a list of top “y” chunks that are relevant to each variant question in the set of variant question at block 308. In one implementation, the chunk re-ranking system uses a cross-encoder model 118 to identify and obtain a list of top “y” chunks (VQ1-Top Y-C, VQ2-Top Y-C, VQ3-Top Y-C . . . ) from the list of top “x” chunks. In certain examples, the number of chunks in the ranked list of top “y” chunks is less than the number of chunks in the ranked list of top “x” chunks.

The cross-encoder model 118 typically consists of multiple layers of neural network units, such as transformers or recurrent neural networks (RNNs), which encodes information from an input it receives into fixed-size representations. In one example, the input provided to the cross-encoder model consists of a variant question and a chunk from the ranked list of top “x” chunks. The input is passed simultaneously to a transformer network within the cross-encoder model. The cross-encoder model then outputs a single score between 0 and 1 indicating the relevance of the chunk to the given variant question. The scores obtained for each chunk are then used by the model to re-rank the list of top “x” chunks to identify a list of top “y” chunks that are relevant to the variant question.

In certain embodiments, the cross-encoder model may be trained using a passage ranking dataset which is a large dataset comprising search questions and their corresponding relevant text passages. For instance, in one example, the cross-encoder model can be trained using a MS MARCO (Microsoft Machine Reading Comprehension) dataset that is focused on machine reading comprehension, question answering, and passage ranking. In other embodiments, a large scale dataset (comprising approximately a million samples) can be constructed and used by the system by mining question and answer (QA) pairs that include both positive and negative examples. For instance, the dataset can be constructed using publically available web crawl data. The positive question pairs can be derived naturally from the QA pairs. The negative question pairs can be generated using a variety of techniques. For instance, in one example, a hard-negative sampling technique can be used where the relevant chunks are collected for each question using a retriever and the chunks that do not answer the question are labelled as negative. In another example, a random negative sampling technique can be used where random chunks are sampled for each question excluding the answer and the chunks retrieved using the hard negative sampling technique.

At block 310, the summary generation system concatenates the list of top “y” chunks identified in 308 to form the content (i.e., generate contextual content) for answering each variant question in the set of variant questions. In a certain implementation, the summary generation system provides the list of top y” chunks (VQ1-Top Y-C, VQ2-Top Y-C, VQ3-Top Y-C . . . ) to a concatenator 120 which concatenates the ranked list of top “y” chunks to form content (i.e., also referred to herein as contextual content) for answering each variant question in the set of variant questions.

At block 312, for each variant question in the set of variant questions, the summary generation system uses the contextual content generated in 310 and the variant question to make a call to a LLM to cause the LLM to generate an answer for the variant question based upon the contextual context. For instance, as depicted in FIG. 1, the concatenator 120 provides the concatenated context (VQ1 context, VQ2 context, VQ3 context . . . ) for answering each variant question in the set of variant questions to LLM-2 122 for further processing. LLM-2 122 then processes the concatenated context for each variant question and generates an answer for each variant question based upon the contextual context.

At block 314, for each question in the set of original questions, the summary generation system combines the answers generated in 312 for variants of the original question to generate a single answer for the original question. For instance, as shown in the embodiment depicted in FIG. 1, the set of answers (VQ-1 A, VQ-2 A, VQ-3 A . . . ) generated for each variant question in the set of variant questions are then provided to an original question answer subsystem 124 within the summary generation system 102 for further processing. The original question answer subsystem 124 generates an answer (OQ1-A, OQ2-A, OQ3-A . . . ) for each original question in the set of original questions by combining the answers generated for each variant question corresponding to the original question.

At block 316, from the answers generated in 314, the summary selection system identifies a selected set of answers by filtering out any text answer that does not contain useful information. For instance, as depicted in FIG. 1, the answer filtration and answer collation subsystem 126 selects a set of answers from the set of answers (OQ1-A, OQ2-A, OQ3-A . . . ) generated for the set of original questions by applying filtering criteria as described in FIG. 3 to the generated answers.

At block 318, the summary generation system collates the answers selected in 316 to generate a summary for the collated result.

At block 320, the summary generation system uses an LLM to generate a summary for the collated result generated in 318. For instance, in the embodiment depicted in FIG. 1, the answer filtration and answer collation subsystem provides the collated result 127 as an input to a LLM (LLM-3 128).

At block 322, the summary generation system provides the summary generated in 320 as the summary for the content to be summarized received in 302 to a requesting user. For instance, as part of generating the summary, in certain examples, the summary generation system may display the summary via a UI of a computing device of the requesting user.

FIG. 4 is a simplified flowchart 400 depicting the processing performed by one or more subsystems in the summary generation system 102 shown in FIG. 1 to identify a list of relevant chunks from content to be summarized, according to certain embodiments. The processing depicted in FIG. 4 may be implemented in software (e.g., code, instructions, program) executed by one or more processing units (e.g., processors, cores) of the respective systems, using hardware, or combinations thereof. The software may be stored on a non-transitory storage medium (e.g., on a memory device). The method presented in FIG. 4 and described below is intended to be illustrative and non-limiting. Although FIG. 4 depicts the various processing steps occurring in a particular sequence or order, this is not intended to be limiting. In certain alternative embodiments, the processing may be performed in some different order, or some steps may also be performed in parallel. In certain embodiments, such as in the embodiment depicted in FIG. 1, the processing depicted in FIG. 4 may be performed by the relevant chunk retrieval subsystem 110, the embeddings generator-1 110, the embeddings generator-2 112 and the index generator 108 described in FIG. 1.

In the embodiment depicted in FIG. 4, processing may be initiated at block 402 when a ML model in the summary generation system 102 obtains a set of chunks from the chunk creation subsystem 104 and processes the set of chunks to generate a summary of each chunk in the set of chunks. The summary generation system 102 may utilize various types of ML models, such as language models (e.g. BERT) and large language models (LLMs) such as GPT-4 to create chunk summaries. In a certain implementation, as depicted in FIG. 1, the multiple chunks (C-1, C-2, C-3 . . . ) created by the chunk creation subsystem 104 are provided to a LLM (LLM-1 106) which then processes each chunk to generate a summary for the chunk. The LLM-1 106 may utilize various input parameters to generate a summary for a chunk. These input parameters may include, for instance, a prompt provided to the LLM, the summarization strategy (e.g., chain of thought prompting technique, a multi-hop question answer evaluation) used by the LLM to generate a summary, a temperature setting used by the LLM to generate the summary and metadata indicating the type of source content used by the LLM to generate the summary.

The prompt is a specific input (e.g., 105) that is provided to the LLM that requests the LLM to perform a specific task (e.g., to generate a summary). For instance, in the context of a medical summary domain, the prompt may represent an input such as “Summarize the following note: \n{hospital_notes}.” The prompt may be automatically generated by a prompt generator (not shown in FIG. 1) within the summary generation system 102 and provided to the LLM for analysis or the prompt can be manually provided by a user of the summary generation system 102 to the LLM. The temperature setting is used to determine how likely the LLM is to come up with different options for the next word during generation of a summary. A higher temperature setting increases the randomness of the generated summary. In a specific implementation, a temperature setting of 0.2 may be used. In other implementations, other values of temperature settings may be used. The metadata indicating the type of source content represents the type of document being used to generate a summary. For instance, in the context of a medical domain, the type of source content can represent an admission note, a progress note, a procedure note, a consultation note, and so on.

Illustrative examples of chunks (C-1, C-2, C-3 . . . ) created by the chunk creation subsystem 104 from a set of documents (for example, the admission note, the progress note, the procedure note, and the consultation note depicted in example 1, example 2, example 3 and example 4 respectively) are shown below. The examples shown below additionally illustrate examples of chunk summaries (SC-1, SC-2, SC-3 . . . ) created for the chunks by an LLM (e.g., LLM-1 106) shown in FIG. 1. The examples shown below are merely illustrations of chunks and chunk summaries created by the summary generation system. One of ordinary skill in the art would recognize many possible variations, alternatives, and modifications. For example, in some implementations, the summary generation system 102 may be configured to create fewer or more chunks and chunk summaries from each document in the set of documents represented in example 1, example 2, example 3, and example 4 described above.

In certain examples, the multiple chunks (C-1, C-2, C-3 . . . ) are created by the chunk creation subsystem 104 by dividing the set of documents to be summarized using a recursive text splitter to create one or more chunks of a particular chunk size (e.g., a paragraph). For instance, example 5 shown below illustrates a chunk (C-1) that is created by the chunk creation subsystem 104 based on the “operative note” document shown in example-1 above. Example 6 illustrates a summary created for C-1 by the LLM. In this example, the chunk creation subsystem 104 creates a single chunk based on the “operative note” document. In alternate examples, however, the chunk creation subsystem 104 may further divide the “operative note” document into additional chunks.

### Example 5: C-1 (Operative Note Document)

Operative Note, 7 Jul. 2023 Patient: John Doe, DOB Jan. 14, 1961, MR 6128394
Procedure: Colonoscopy
Indication: Evaluation of lower GI bleed
Findings: Colonoscopy showed diverticulosis in the sigmoid colon with evidence of recent bleeding.
Procedure: Colonoscopy was performed under conscious sedation. The scope was advanced to the cecum. The co Ion was carefully examined on withdrawal. There was evidence of diverticulosis in the sigmoid colon. A large clot was noted in one of the diverticula, but no active bleeding was identified.
Impression:
1. Diverticulosis in the sigmoid colon with evidence of recent bleeding
Plan:
1. Diverticular bleeding: The patient should follow a high fiber diet to prevent further episodes of diverticular bleeding.
2. Continue IV fluids and monitor hemoglobin closely.
3. Plan for discharge once stable

### Example 6: Summary for C-1 (SC-1)

John Doe, born on Jan. 14, 1961, underwent a colonoscopy on 7 Jul. 2023 to evaluate lower GI bleeding. The procedure revealed diverticulosis in the sigmoid colon with evidence of recent bleeding. No active bleeding was identified, but a large clot was found in one of the diverticula. The patient is advised to follow a high fiber diet to prevent further episodes of diverticular bleeding, continue IV fluids, monitor hemoglobin closely, and plan for discharge once stable.

Example 7 shown below illustrates a chunk (C-2) that is created by the chunk creation subsystem 104 based on the “progress note” document shown in example 2 above. Example 8 illustrates a summary created for C-2 by the LLM.

### Example 7: C-2 (Progress Note)

Progress Note, 8 Jul. 2023 Patient: John Doe, DOB Jan. 14, 1961, MR 6128394
S: The patient reports no further episodes of rectal bleeding. Denies abdominal pain.
O: ⋅BP 130/75, HR 80, Temp 98.6° F., RR 16, SpO2 98% Physical Exam: ⋅General: Appears comfortable ⋅Cardiovascular: Regular rhythm, no murnmurs ⋅Respiratory: Clear to auscultation bilaterally ⋅Abdomen: Soft, non-ten der ⋅Rectal: No blood on glove Labs: ⋅Hgb: 10.6 g/dL
A:
1. Diverticular Bleeding: Stable, no further bleeding episodes
2. Hypertension: Controlled
3. Hyperlipidemia: Controlled
P:
1. Continue with IV hydration.
2. Monitor hemoglobin.
3. Re-introduce solid food as tolerated
S: The patient reports feeling well, tolerating diet.
O: ⋅BP 125/80, HR 78, Temp 98.4° F., RR 16, SpO2 98% Physical Exam: ⋅General: No acute distress ⋅Cardiovascular: Regular rhythm, no murmurs ⋅Respiratory: Clear to auscultation bilaterally ⋅Abdomen: Soft, non-tender ⋅Rectal: No blood on glove Labs: ⋅Hgb: 11.0 g/dL
A:
1. Diverticular Bleeding: Stable, no further bleeding episodes
2. Hypertension: Controlled
3. Hyperlipidemia: Controlled
P:
1. Discontinue IV fluids.
2. Continue to monitor hemoglobin.
3. Plan for discharge tomorrow if remains stable.
Progress Note, 10 Jul. 2023 Patient: John Doe, DOB Jan. 14, 1961, MR 6128394
S: The patient reports feeling well, no episodes of rectal bleeding. Tolerating diet.

### Example 8: Summary for C-2 (SC-2)

John Doe, a patient with diverticular bleeding, hypertension, and hyperlipidemia, has been stable with no further episodes of rectal bleeding. His vital signs are within normal limits, and his hemoglobin levels have improved. He is tolerating his diet well and is planned for discharge the following day if he remains stable.

In certain examples, the chunk creation subsystem 104 may divide a particular document into multiple chunks. For instance, the “progress note” document may be divided into two chunks C-2 and C-3 as shown in example 9 below. Example 10 illustrates a summary created for C-3 by the LLM.

### Example 9: C-3 (Progress Note)

Progress Note, 10 Jul. 2023 Patient: John Doe, DOB Jan. 14, 1961, MR 6128394
S: The patient reports feeling well, no episodes of rectal bleeding. Tolerating diet.
O: ⋅BP 120/78, HR 77, Temp 98.2° F., RR 16, SpO2 98% Physical Exam: ⋅General: No acute distress ⋅Cardiovascular: Regular rhythm, no murmurs ⋅Respiratory: Clear to auscultation bilaterally. Abdomen Soft, non-tender ⋅
Rectal: No blood on glove Labs: ⋅Hgb: 11.2 g/dL
A:
1. Diverticular Bleeding: Stable, no further bleeding episodes
2. Hypertension: Controlled
3. Hyperlipidemia: Controlled
P:
1. Patient is stable for discharge.
2. Recommend follow-up with a gastroenterologist in 1-2 weeks.
3. Recommend a high fiber diet to help prevent further episodes of diverticular bleeding.
4. Continue home medications.
5. Patient to return to ER if bleeding recurs or if he experiences any severe abdominal pain, fever, or other new symptoms.

### Example 10: Summary for C-3 (SC-3)

John Doe, a 62-year-old patient, presented feeling well with no rectal bleeding episodes. Vital signs were within normal limits, physical exam was unremarkable, and labs showed stable hemoglobin levels. Diagnoses of diverticular bleeding, hypertension, and hyperlipidemia were controlled. Patient is stable for discharge with recommendations for follow-up with a gastroenterologist, high fiber diet, and continuation of home medications. Patient advised to return to ER if bleeding recurs or new symptoms develop.

The examples 11-18 illustrated below depict additional chunks created by the chunk creation system based on the documents represented in examples 1-4. For instance, example 11 shown below illustrates a chunk (C-4) that is created by the chunk creation subsystem 104 from the “admission note” document. Example 12 illustrates a summary created for C-4 by the LLM.

### Example 11: C-4 (Admission Note)

History and Physical Note, 6 Jul. 2023 Patient: John Doe, DOB Jan. 14, 1961, MR 6128394
HPI: John Doe is a 62-year-old male with a past medical history of hypertension and hyperlipidemia who presents with a 24-hour history of rectal bleeding. He reports that the bleeding started suddenly yesterday evening, describing the blood as bright red and mixed with stool. He also reports experiencing three episodes of such bleeding, each associated with bowel movements. The amount of blood is significant, enough to turn the toilet water red. He denies any associated abdominal pain, nausea, vomiting, or changes in bowel habits. The patient has never experienced anything similar in the past. He denies any history of peptic ulcer disease, gastritis, or gastrointestinal malignancies. He also denies any recent changes in his diet, alcohol use, or NSAID use. The patient sought care when he noticed a drop in his energy level. He reported feeling light-headed and slightly short of breath during minimal exertion. He denies chest pain, palpitations, or syncope. He also denies fever, night sweats, weight loss, or other systemic symptoms.
The patient's past medical history is significant for hypertension, which is well controlled with Lisinopril, and hyperlipidemia, managed with Atorvastatin. He reports medication compliance and has not made any recent changes to his medication regimen. He denies tobacco use and uses alcohol occasionally, in moderation. There is no known family history of colorectal cancer or inflammatory bowel disease. He has never had a colonoscopy.

### Example 12: Summary for C-4 (SC-4)

John Doe, a 62-year-old male with a history of hypertension and hyperlipidemia, presents with a 24-hour history of significant rectal bleeding. He denies abdominal pain, nausea, vomiting, or changes in bowel habits. He reports feeling light-headed and short of breath with minimal exertion. His past medical history includes well-controlled hypertension and hyperlipidemia. He denies tobacco use and has occasional, moderate alcohol consumption. Family history is negative for colorectal cancer or inflammatory bowel disease. He has never had a colonoscopy.

Example 13 shown below illustrates another chunk (C-5) that is created by the chunk creation subsystem 104 from the “admission note” document. Example 14 illustrates a summary created for C-5 by the LLM.

### Example 13: C-5 (Admission Note)

Upon arrival to the emergency department, vital signs showed blood pressure of 110/70 mmHg, heart rate of 100 beats per minute, respiratory rate of 16 breaths per minute, body temperature of 98.6° F., and oxygen saturation of 97% on room air. Physical examination revealed pale conjunctiva and mild tachycardia but was otherwise unremarkable, including a benign abdominal exam. Initial lab tests showed a drop in hemoglobin from his baseline of 14 g/dL to 9 g/dL. Liver function tests, including coagulation parameters, were within normal limits. Given his presentation and these findings, he was admitted to the hospital for further evaluation and management of a lower GI bleed.
Past Medical History: ⋅Hypertension ⋅Hyperlipidemia
Past Surgical History: ⋅Appendectomy in 1983
Social History: Mr. Doe is a retired engineer, married, with 2 grown-up children. He is a lifelong non-smoker and denies illicit drug use. He reports social alcohol use, with an average of 1-2 drinks per week.
Home Medications: ⋅Lisinopril 10 mg daily ⋅Atorvastatin 20 mg daily
Physical Exam: ⋅General—Patient appears comfortable, in no acute distress ⋅HEENT—Normocephalic atraumatic, mucus membranes moist ⋅Cardiovascular—RRR, no murmurs ⋅Respiratory—Clear to auscultation bilaterally ⋅Abdomen—Soft, non-tender, non-distended, no palpable masses or hepatosplenomegaly ⋅Rectal—Bright red blood on glove, no palpable masses, normal sphincter tone ⋅Extremities—Warm and well perfused, no edema
Labs: ⋅CBC: WBC 8.2, Hgb 11.0, Pit 210 ⋅BMP: Na 139, K 4.0, Cl 104, CO2 26, BUN 18, Cr 0.9, Glucose 102 ⋅Coagulation Profile: PT 11.5, INR 1.1, aPTT 30
Assessment & Plan:

### Example 14: Summary for C-5 (SC-5)

A 65-year-old male with a history of hypertension and hyperlipidemia presented to the emergency department with symptoms of lower GI bleed. Vital signs were stable, but physical examination revealed pale conjunctiva and mild tachycardia. Initial lab tests showed a drop in hemoglobin levels. He was admitted to the hospital for further evaluation and management. Past medical history includes an appendectomy in 1983, and he is currently on lisinopril and atorvastatin. Social history is significant for social alcohol use. Physical exam was unremarkable except for bright red blood on rectal exam. Labs showed mild anemia and normal liver function tests

Example 15 shown below illustrates yet another chunk (C-6) that is created by the chunk creation subsystem 104 from the “admission note” document. Example 16 illustrates a summary created for the chunk 5 by the LLM.

### Example 15: C-5 (Admission Note)

Labs: ⋅CBC: WBC 8.2, Hgb 11.0, Pit 210 ⋅BMP: Na 139, K 4.0, Cl 104, CO2 26, BUN 18, Cr 0.9, Glucose 102 ⋅Coagulation Profile: PT 11.5, INR 1.1, aPTT 30
Assessment & Plan:
1. Lower GI Bleed: Start IV hydration, transfuse 2 units of packed RBCs, arrange for urgent gastroenterology consult for colonoscopy. Maintain NPO status.
2. Hypertension: Continue with Lisinopril.
3. Hyperlipidemia: Continue with Atorvastatin.

A gastroenterology consult will be obtained for further management.

### Example 16: Summary for C-5 (SC-5)

The patient has a lower GI bleed and will receive IV hydration, 2 units of packed RBCs, and a gastroenterology consult for a colonoscopy. They also have hypertension and hyperlipidemia, for which they will continue Lisinopril and Atorvastatin, respectively.

Example 17 shown below illustrates a chunk (C-6) that is created by the chunk creation subsystem 104 based on the “consultation note” document shown in example 4 above. Example 18 illustrates a summary created for C-6 by the LLM.

### Example 16: C-6 (Admission Note)

Gastroenterology Consultation Note, 7 Jul. 2023 Patient: John Doe, DOB Jan. 14, 1961, MR 6128394
Reason for consult: Lower GI Bleed
History of Present Illness: Mr. Doe is a 62-year-old male with a history of hypertension and hyperlipidemia, presented with the complaint of bright red blood per rectum. He reports the symptoms started two days ago with intermittent lower abdominal cramping. There's no known history of GI bleeds, peptic ulcer disease, or liver disease. He denies any recent NSAID or anticoagulant use.
Physical Exam: ⋅General: No acute distress ⋅Cardiovascular: Regular rhythm, no murmurs ⋅Respiratory: Clear to auscultation bilaterally ⋅Abdomen: Soft, non-tender ⋅Rectal: Bright red blood on glove Laboratory data: ⋅Hgb: 11.0 g/dL (lower from baseline of 14 g/dL) ⋅Coagulation Profile: PT 11.5, INR 1.1, aPTT 30
Assessment:
1. Lower GI bleed: Mr. Doe's presentation of bright red blood per rectum, coupled with a drop in hemoglobin, is consistent with a lower gastrointestinal bleed. The absence of any significant upper GI symptoms and normal liver function tests rules out an upper GI source.
Plan:
1. Colonoscopy: Will schedule an urgent colonoscopy for further evaluation and to identify the source of bleeding.
2. Resuscitation: Continue IV fluids and transfusion of packed red blood cells.
3. GI prophylaxis: Start proton pump inhibitor for gastric mucosa protection.
4. Recheck Labs: Monitor hemoglobin and vital signs closely.

### Example 17: Summary for C-6 (SC-6)

62-year-old male, John Doe, presented with bright red blood per rectum and lower abdominal cramping. No history of GI bleeds, peptic ulcer disease, or liver disease. Physical exam and lab data support a lower GI bleed. Plan includes urgent colonoscopy, resuscitation with IV fluids and blood transfusion, GI prophylaxis, and close monitoring of labs and vital signs.

After creating multiple chunks and multiple chunk summaries as described above, the summary generation system 102 identifies and retrieves a first subset of relevant chunks (i.e., a list of top “x” chunks) from the multiple chunks for each variant question in the set of variant questions. In a certain implementation, a relevant chunk retrieval subsystem 110 within the summary generation system 102 uses a first technique to identify and retrieve the first subset of chunks that are relevant to (i.e., answered by) each variant question in the set of variant questions. The first technique involves generating embeddings for the multiple chunks and generating embeddings for the multiple summaries generated for the multiple chunks. The technique uses the embeddings to rank the multiple chunks based on relevance of each chunk in the multiple chunks to the variant question. The technique then involves selecting a first subset of chunks (i.e., a list of top “x” chunks) that are relevant to the variant question based on the ranking.

As part of the processing to identify a first subset of relevant chunks using the first technique, the relevant chunk retrieval subsystem 110 generates two embeddings for each chunk: (1) an embedding for the content in the chunk and (2) an embedding for the summary generated for the chunk in 402. The chunk embeddings and the chunk summary embeddings may represent a set of vectors which may be phrases, clauses, or other segments of text that contain multiple words in the chunk or the summary of the chunk that help capture the contextual meaning of a group of words in a more efficient way, as opposed to treating each word separately. In some examples, the chunk embeddings and the chunk summary embeddings may represent high-dimensional numeric vectors that can be used to measure semantic similarity between words or sentences in the chunks and chunk summaries. In a certain implementation, and as depicted in FIG. 1, the embedding for the content in each chunk is generated by the embeddings generator-1 110 within the summary generation system 102. The embedding for the summary for each chunk is generated by the embeddings generator-1 112 within the summary generation system 102.

In certain embodiments, at block 406, the relevant chunk retrieval subsystem 110 additionally generates an inverted search index from the content to be summarized. In a certain implementation, the inverted search index is generated by the index generator shown in FIG. 1 using a ranking function. In one example, the ranking function is a BM25 (Best Match 25) function that scores each document in a set of documents representing the content to be summarized by determining the frequency of the appearance of a particular word in each of the documents.

At block 406, the relevant chunk retrieval subsystem 110 ranks the multiple chunks based on relevance of each chunk in the multiple chunks to the variant question using the embeddings created for chunks, the embeddings created for the chunk summaries, and the inverted index. The subsystem then identifies and retrieves a list of top “x” chunks that are relevant to each variant question in the set of variant questions defined in the reference information 112 based on the ranking. The chunk retrieval subsystem 110 then provides the list of top “x” chunks to the chunk re-ranking subsystem 116 as described in FIG. 3 to identify and retrieve a list of top “y” chunks (i.e., a second subset of chunks) that are relevant to each variant question in the set of variant questions.

As described above, the re-ranking of the chunks is performed to ensure that chunks of utmost relevance are identified and retrieved. As part of the processing performed in this stage, the system additionally utilizes a set of comprehensive questions defined by subject matter experts to further guide the retrieval of relevant chunks of information. Each question undergoes a translation or transformation process to generate multiple question variants to optimize the search to obtain relevant chunks of information. The system uses the list of top “y” chunks and a large language model (LLM) to extract relevant information (i.e., answers) from the set of questions. In certain examples, and as described above, the system utilizes the multiple variants for each question to generate answers from the relevant chunks of information.

The system then collates the answers and uses a collated result (answer) to create an accurate and comprehensive summary for the content to be summarized. The results of the processing performed by the system 102 are then communicated back to the requesting user. These results may include a summary (e.g., 130) for the content to be summarized. For instance, if the content to be summarized represents a set of documents related to a patient as described above, the results generated by the system may include a hospital discharge summary generated for the patient as described above.

As noted above, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (e.g., billing, monitoring, logging, load balancing and clustering, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

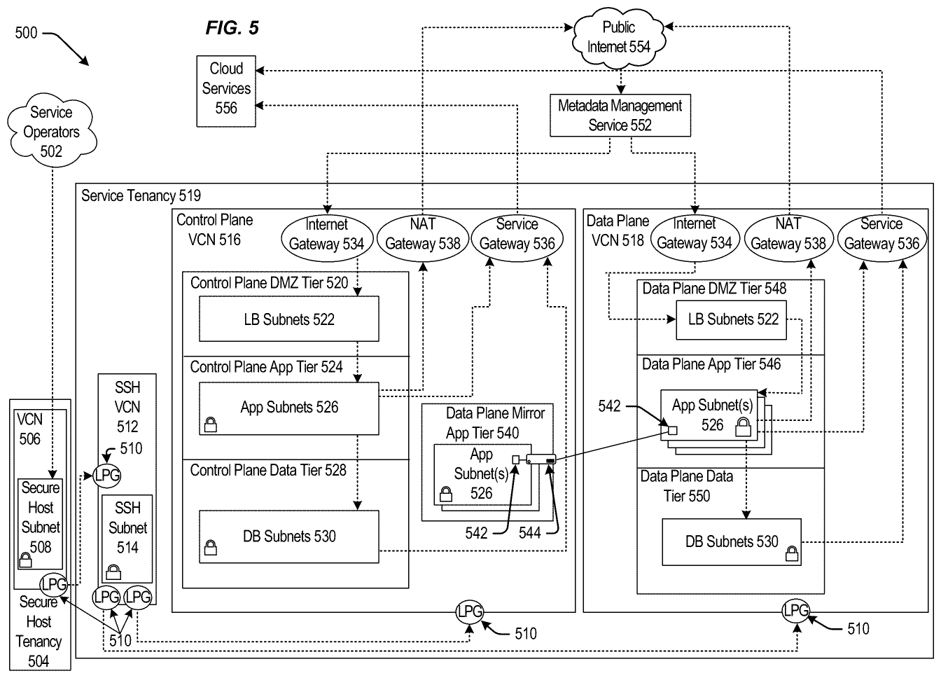

FIG. 5 is a block diagram 500 illustrating an example pattern of an IaaS architecture, according to at least one embodiment. Service operators 502 can be communicatively coupled to a secure host tenancy 504 that can include a virtual cloud network (VCN) 506 and a secure host subnet 508. In some examples, the service operators 502 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 506 and/or the Internet.

The VCN 506 can include a local peering gateway (LPG) 510 that can be communicatively coupled to a secure shell (SSH) VCN 512 via an LPG 510 contained in the SSH VCN 512. The SSH VCN 512 can include an SSH subnet 514, and the SSH VCN 512 can be communicatively coupled to a control plane VCN 516 via the LPG 510 contained in the control plane VCN 516. Also, the SSH VCN 512 can be communicatively coupled to a data plane VCN 518 via an LPG 510. The control plane VCN 516 and the data plane VCN 518 can be contained in a service tenancy 519 that can be owned and/or operated by the IaaS provider.

The control plane VCN 516 can include a control plane demilitarized zone (DMZ) tier 520 that acts as a perimeter network (e.g., portions of a corporate network between the corporate intranet and external networks). The DMZ-based servers may have restricted responsibilities and help keep breaches contained. Additionally, the DMZ tier 520 can include one or more load balancer (LB) subnet(s) 522, a control plane app tier 524 that can include app subnet(s) 526, a control plane data tier 528 that can include database (DB) subnet(s) 530 (e.g., frontend DB subnet(s) and/or backend DB subnet(s)). The LB subnet(s) 522 contained in the control plane DMZ tier 520 can be communicatively coupled to the app subnet(s) 526 contained in the control plane app tier 524 and an Internet gateway 534 that can be contained in the control plane VCN 516, and the app subnet(s) 526 can be communicatively coupled to the DB subnet(s) 530 contained in the control plane data tier 528 and a service gateway 536 and a network address translation (NAT) gateway 538. The control plane VCN 516 can include the service gateway 536 and the NAT gateway 538.

The control plane VCN 516 can include a data plane mirror app tier 540 that can include app subnet(s) 526. The app subnet(s) 526 contained in the data plane mirror app tier 540 can include a virtual network interface controller (VNIC) 542 that can execute a compute instance 544. The compute instance 544 can communicatively couple the app subnet(s) 526 of the data plane mirror app tier 540 to app subnet(s) 526 that can be contained in a data plane app tier 546.

The data plane VCN 518 can include the data plane app tier 546, a data plane DMZ tier 548, and a data plane data tier 550. The data plane DMZ tier 548 can include LB subnet(s) 522 that can be communicatively coupled to the app subnet(s) 526 of the data plane app tier 546 and the Internet gateway 534 of the data plane VCN 518. The app subnet(s) 526 can be communicatively coupled to the service gateway 536 of the data plane VCN 518 and the NAT gateway 538 of the data plane VCN 518. The data plane data tier 550 can also include the DB subnet(s) 530 that can be communicatively coupled to the app subnet(s) 526 of the data plane app tier 546.

The Internet gateway 534 of the control plane VCN 516 and of the data plane VCN 518 can be communicatively coupled to a metadata management service 552 that can be communicatively coupled to public Internet 554. Public Internet 554 can be communicatively coupled to the NAT gateway 538 of the control plane VCN 516 and of the data plane VCN 518. The service gateway 536 of the control plane VCN 516 and of the data plane VCN 518 can be communicatively couple to cloud services 556.

In some examples, the service gateway 536 of the control plane VCN 516 or of the data plane VCN 518 can make application programming interface (API) calls to cloud services 556 without going through public Internet 554. The API calls to cloud services 556 from the service gateway 536 can be one-way: the service gateway 536 can make API calls to cloud services 556, and cloud services 556 can send requested data to the service gateway 536. But, cloud services 556 may not initiate API calls to the service gateway 536.

In some examples, the secure host tenancy 504 can be directly connected to the service tenancy 519, which may be otherwise isolated. The secure host subnet 508 can communicate with the SSH subnet 514 through an LPG 510 that may enable two-way communication over an otherwise isolated system. Connecting the secure host subnet 508 to the SSH subnet 514 may give the secure host subnet 508 access to other entities within the service tenancy 519.

The control plane VCN 516 may allow users of the service tenancy 519 to set up or otherwise provision desired resources. Desired resources provisioned in the control plane VCN 516 may be deployed or otherwise used in the data plane VCN 518. In some examples, the control plane VCN 516 can be isolated from the data plane VCN 518, and the data plane mirror app tier 540 of the control plane VCN 516 can communicate with the data plane app tier 546 of the data plane VCN 518 via VNICs 542 that can be contained in the data plane mirror app tier 540 and the data plane app tier 546.

In some examples, users of the system, or customers, can make requests, for example create, read, update, or delete (CRUD) operations, through public Internet 554 that can communicate the requests to the metadata management service 552. The metadata management service 552 can communicate the request to the control plane VCN 516 through the Internet gateway 534. The request can be received by the LB subnet(s) 522 contained in the control plane DMZ tier 520. The LB subnet(s) 522 may determine that the request is valid, and in response to this determination, the LB subnet(s) 522 can transmit the request to app subnet(s) 526 contained in the control plane app tier 524. If the request is validated and requires a call to public Internet 554, the call to public Internet 554 may be transmitted to the NAT gateway 538 that can make the call to public Internet 554. Metadata that may be desired to be stored by the request can be stored in the DB subnet(s) 530.

In some examples, the data plane mirror app tier 540 can facilitate direct communication between the control plane VCN 516 and the data plane VCN 518. For example, changes, updates, or other suitable modifications to configuration may be desired to be applied to the resources contained in the data plane VCN 518. Via a VNIC 542, the control plane VCN 516 can directly communicate with, and can thereby execute the changes, updates, or other suitable modifications to configuration to, resources contained in the data plane VCN 518.

In some embodiments, the control plane VCN 516 and the data plane VCN 518 can be contained in the service tenancy 519. In this case, the user, or the customer, of the system may not own or operate either the control plane VCN 516 or the data plane VCN 518. Instead, the IaaS provider may own or operate the control plane VCN 516 and the data plane VCN 518, both of which may be contained in the service tenancy 519. This embodiment can enable isolation of networks that may prevent users or customers from interacting with other users', or other customers', resources. Also, this embodiment may allow users or customers of the system to store databases privately without needing to rely on public Internet 554, which may not have a desired level of threat prevention, for storage.

In other embodiments, the LB subnet(s) 522 contained in the control plane VCN 516 can be configured to receive a signal from the service gateway 536. In this embodiment, the control plane VCN 516 and the data plane VCN 518 may be configured to be called by a customer of the IaaS provider without calling public Internet 554. Customers of the IaaS provider may desire this embodiment since database(s) that the customers use may be controlled by the IaaS provider and may be stored on the service tenancy 519, which may be isolated from public Internet 554.

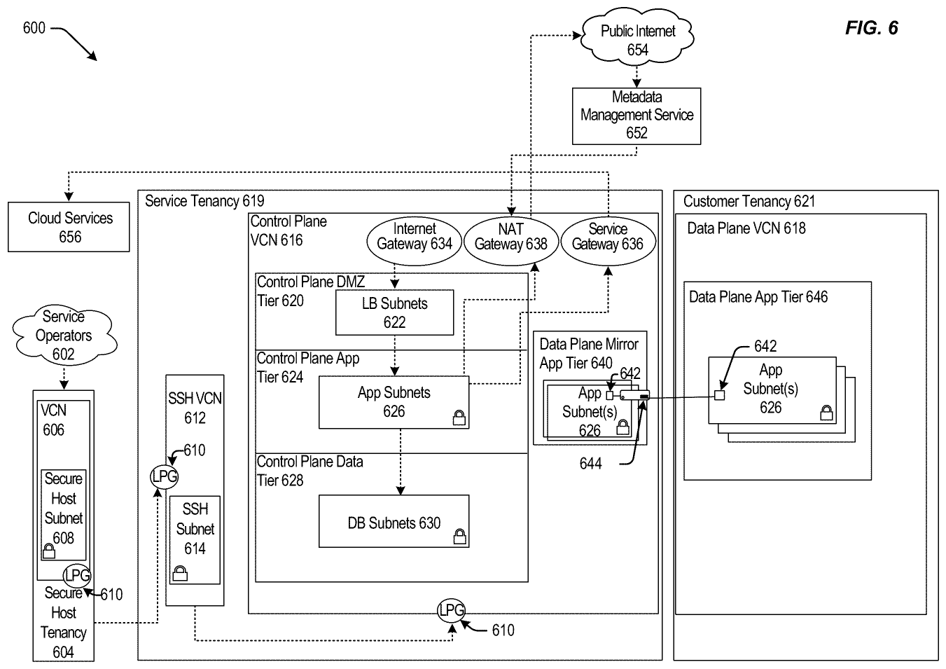

FIG. 6 is a block diagram 600 illustrating another example pattern of an IaaS architecture, according to at least one embodiment. Service operators 602 (e.g., service operators 502 of FIG. 5) can be communicatively coupled to a secure host tenancy 604 (e.g., the secure host tenancy 504 of FIG. 5) that can include a virtual cloud network (VCN) 606 (e.g., the VCN 506 of FIG. 5) and a secure host subnet 608 (e.g., the secure host subnet 508 of FIG. 5). The VCN 606 can include a local peering gateway (LPG) 610 (e.g., the LPG 510 of FIG. 5) that can be communicatively coupled to a secure shell (SSH) VCN 612 (e.g., the SSH VCN 512 of FIG. 5) via an LPG 510 contained in the SSH VCN 612. The SSH VCN 612 can include an SSH subnet 614 (e.g., the SSH subnet 514 of FIG. 5), and the SSH VCN 612 can be communicatively coupled to a control plane VCN 616 (e.g., the control plane VCN 516 of FIG. 5) via an LPG 610 contained in the control plane VCN 616. The control plane VCN 616 can be contained in a service tenancy 619 (e.g., the service tenancy 519 of FIG. 5), and the data plane VCN 618 (e.g., the data plane VCN 518 of FIG. 5) can be contained in a customer tenancy 621 that may be owned or operated by users, or customers, of the system.

The control plane VCN 616 can include a control plane DMZ tier 620 (e.g., the control plane DMZ tier 520 of FIG. 5) that can include LB subnet(s) 622 (e.g., LB subnet(s) 522 of FIG. 5), a control plane app tier 624 (e.g., the control plane app tier 524 of FIG. 5) that can include app subnet(s) 626 (e.g., app subnet(s) 526 of FIG. 5), a control plane data tier 628 (e.g., the control plane data tier 528 of FIG. 5) that can include database (DB) subnet(s) 630 (e.g., similar to DB subnet(s) 530 of FIG. 5). The LB subnet(s) 622 contained in the control plane DMZ tier 620 can be communicatively coupled to the app subnet(s) 626 contained in the control plane app tier 624 and an Internet gateway 634 (e.g., the Internet gateway 534 of FIG. 5) that can be contained in the control plane VCN 616, and the app subnet(s) 626 can be communicatively coupled to the DB subnet(s) 630 contained in the control plane data tier 628 and a service gateway 636 (e.g., the service gateway 536 of FIG. 5) and a network address translation (NAT) gateway 638 (e.g., the NAT gateway 538 of FIG. 5). The control plane VCN 616 can include the service gateway 636 and the NAT gateway 638.

The control plane VCN 616 can include a data plane mirror app tier 640 (e.g., the data plane mirror app tier 540 of FIG. 5) that can include app subnet(s) 626. The app subnet(s) 626 contained in the data plane mirror app tier 640 can include a virtual network interface controller (VNIC) 642 (e.g., the VNIC of 542) that can execute a compute instance 644 (e.g., similar to the compute instance 544 of FIG. 5). The compute instance 644 can facilitate communication between the app subnet(s) 626 of the data plane mirror app tier 640 and the app subnet(s) 626 that can be contained in a data plane app tier 646 (e.g., the data plane app tier 546 of FIG. 5) via the VNIC 642 contained in the data plane mirror app tier 640 and the VNIC 642 contained in the data plane app tier 646.

The Internet gateway 634 contained in the control plane VCN 616 can be communicatively coupled to a metadata management service 652 (e.g., the metadata management service 552 of FIG. 5) that can be communicatively coupled to public Internet 654 (e.g., public Internet 554 of FIG. 5). Public Internet 654 can be communicatively coupled to the NAT gateway 638 contained in the control plane VCN 616. The service gateway 636 contained in the control plane VCN 616 can be communicatively couple to cloud services 656 (e.g., cloud services 556 of FIG. 5).

In some examples, the data plane VCN 618 can be contained in the customer tenancy 621. In this case, the IaaS provider may provide the control plane VCN 616 for each customer, and the IaaS provider may, for each customer, set up a unique compute instance 644 that is contained in the service tenancy 619. Each compute instance 644 may allow communication between the control plane VCN 616, contained in the service tenancy 619, and the data plane VCN 618 that is contained in the customer tenancy 621. The compute instance 644 may allow resources, that are provisioned in the control plane VCN 616 that is contained in the service tenancy 619, to be deployed or otherwise used in the data plane VCN 618 that is contained in the customer tenancy 621.

In other examples, the customer of the IaaS provider may have databases that live in the customer tenancy 621. In this example, the control plane VCN 616 can include the data plane mirror app tier 640 that can include app subnet(s) 626. The data plane mirror app tier 640 can reside in the data plane VCN 618, but the data plane mirror app tier 640 may not live in the data plane VCN 618. That is, the data plane mirror app tier 640 may have access to the customer tenancy 621, but the data plane mirror app tier 640 may not exist in the data plane VCN 618 or be owned or operated by the customer of the IaaS provider. The data plane mirror app tier 640 may be configured to make calls to the data plane VCN 618 but may not be configured to make calls to any entity contained in the control plane VCN 616. The customer may desire to deploy or otherwise use resources in the data plane VCN 618 that are provisioned in the control plane VCN 616, and the data plane mirror app tier 640 can facilitate the desired deployment, or other usage of resources, of the customer.

In some embodiments, the customer of the IaaS provider can apply filters to the data plane VCN 618. In this embodiment, the customer can determine what the data plane VCN 618 can access, and the customer may restrict access to public Internet 654 from the data plane VCN 618. The IaaS provider may not be able to apply filters or otherwise control access of the data plane VCN 618 to any outside networks or databases. Applying filters and controls by the customer onto the data plane VCN 618, contained in the customer tenancy 621, can help isolate the data plane VCN 618 from other customers and from public Internet 654.

In some embodiments, cloud services 656 can be called by the service gateway 636 to access services that may not exist on public Internet 654, on the control plane VCN 616, or on the data plane VCN 618. The connection between cloud services 656 and the control plane VCN 616 or the data plane VCN 618 may not be live or continuous. Cloud services 656 may exist on a different network owned or operated by the IaaS provider. Cloud services 656 may be configured to receive calls from the service gateway 636 and may be configured to not receive calls from public Internet 654. Some cloud services 656 may be isolated from other cloud services 656, and the control plane VCN 616 may be isolated from cloud services 656 that may not be in the same region as the control plane VCN 616. For example, the control plane VCN 616 may be located in “Region 1,” and cloud service “Deployment 5,” may be located in Region 1 and in “Region 2.” If a call to Deployment 5 is made by the service gateway 636 contained in the control plane VCN 616 located in Region 1, the call may be transmitted to Deployment 5 in Region 1. In this example, the control plane VCN 616, or Deployment 5 in Region 1, may not be communicatively coupled to, or otherwise in communication with, Deployment 5 in Region 2.

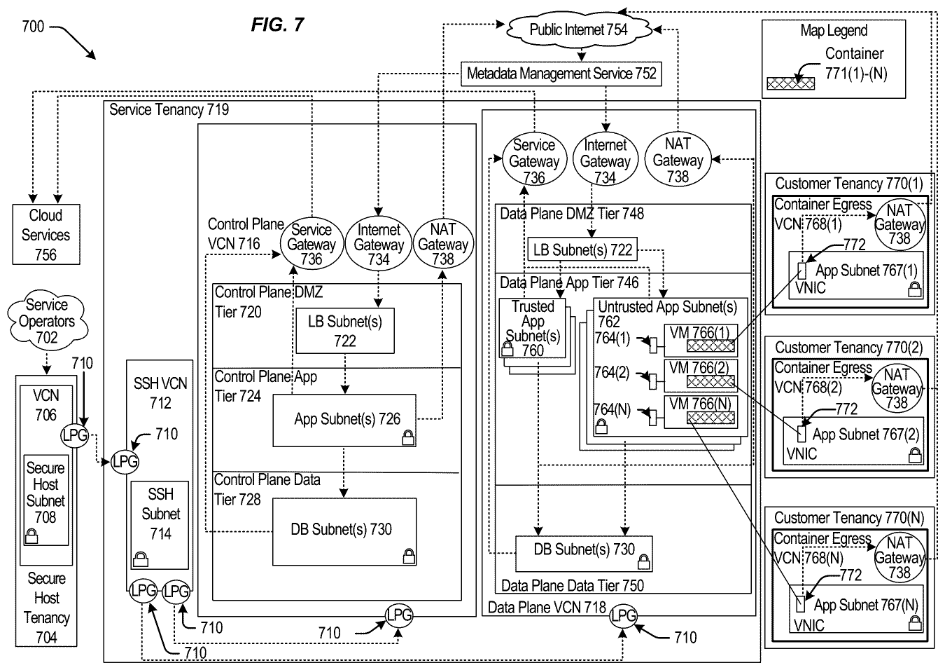

FIG. 7 is a block diagram 700 illustrating another example pattern of an IaaS architecture, according to at least one embodiment. Service operators 702 (e.g., service operators 502 of FIG. 5) can be communicatively coupled to a secure host tenancy 704 (e.g., the secure host tenancy 504 of FIG. 5) that can include a virtual cloud network (VCN) 706 (e.g., the VCN 506 of FIG. 5) and a secure host subnet 708 (e.g., the secure host subnet 508 of FIG. 5). The VCN 706 can include an LPG 710 (e.g., the LPG 510 of FIG. 5) that can be communicatively coupled to an SSH VCN 712 (e.g., the SSH VCN 512 of FIG. 5) via an LPG 710 contained in the SSH VCN 712. The SSH VCN 712 can include an SSH subnet 714 (e.g., the SSH subnet 514 of FIG. 5), and the SSH VCN 712 can be communicatively coupled to a control plane VCN 716 (e.g., the control plane VCN 516 of FIG. 5) via an LPG 710 contained in the control plane VCN 716 and to a data plane VCN 718 (e.g., the data plane 518 of FIG. 5) via an LPG 710 contained in the data plane VCN 718. The control plane VCN 716 and the data plane VCN 718 can be contained in a service tenancy 719 (e.g., the service tenancy 519 of FIG. 5).

The control plane VCN 716 can include a control plane DMZ tier 720 (e.g., the control plane DMZ tier 520 of FIG. 5) that can include load balancer (LB) subnet(s) 722 (e.g., LB subnet(s) 522 of FIG. 5), a control plane app tier 724 (e.g., the control plane app tier 524 of FIG. 5) that can include app subnet(s) 726 (e.g., similar to app subnet(s) 526 of FIG. 5), a control plane data tier 728 (e.g., the control plane data tier 528 of FIG. 5) that can include DB subnet(s) 730. The LB subnet(s) 722 contained in the control plane DMZ tier 720 can be communicatively coupled to the app subnet(s) 726 contained in the control plane app tier 724 and to an Internet gateway 734 (e.g., the Internet gateway 534 of FIG. 5) that can be contained in the control plane VCN 716, and the app subnet(s) 726 can be communicatively coupled to the DB subnet(s) 730 contained in the control plane data tier 728 and to a service gateway 736 (e.g., the service gateway of FIG. 5) and a network address translation (NAT) gateway 738 (e.g., the NAT gateway 538 of FIG. 5). The control plane VCN 716 can include the service gateway 736 and the NAT gateway 738.

The data plane VCN 718 can include a data plane app tier 746 (e.g., the data plane app tier 546 of FIG. 5), a data plane DMZ tier 748 (e.g., the data plane DMZ tier 548 of FIG. 5), and a data plane data tier 750 (e.g., the data plane data tier 550 of FIG. 5). The data plane DMZ tier 748 can include LB subnet(s) 722 that can be communicatively coupled to trusted app subnet(s) 760 and untrusted app subnet(s) 762 of the data plane app tier 746 and the Internet gateway 734 contained in the data plane VCN 718. The trusted app subnet(s) 760 can be communicatively coupled to the service gateway 736 contained in the data plane VCN 718, the NAT gateway 738 contained in the data plane VCN 718, and DB subnet(s) 730 contained in the data plane data tier 750. The untrusted app subnet(s) 762 can be communicatively coupled to the service gateway 736 contained in the data plane VCN 718 and DB subnet(s) 730 contained in the data plane data tier 750. The data plane data tier 750 can include DB subnet(s) 730 that can be communicatively coupled to the service gateway 736 contained in the data plane VCN 718.

The untrusted app subnet(s) 762 can include one or more primary VNICs 764(1)-(N) that can be communicatively coupled to tenant virtual machines (VMs) 766(1)-(N). Each tenant VM 766(1)-(N) can be communicatively coupled to a respective app subnet 767(1)-(N) that can be contained in respective container egress VCNs 768(1)-(N) that can be contained in respective customer tenancies 770(1)-(N). Respective secondary VNICs 772(1)-(N) can facilitate communication between the untrusted app subnet(s) 762 contained in the data plane VCN 718 and the app subnet contained in the container egress VCNs 768(1)-(N). Each container egress VCNs 768(1)-(N) can include a NAT gateway 738 that can be communicatively coupled to public Internet 754 (e.g., public Internet 554 of FIG. 5).

The Internet gateway 734 contained in the control plane VCN 716 and contained in the data plane VCN 718 can be communicatively coupled to a metadata management service 752 (e.g., the metadata management system 552 of FIG. 5) that can be communicatively coupled to public Internet 754. Public Internet 754 can be communicatively coupled to the NAT gateway 738 contained in the control plane VCN 716 and contained in the data plane VCN 718. The service gateway 736 contained in the control plane VCN 716 and contained in the data plane VCN 718 can be communicatively couple to cloud services 756.

In some embodiments, the data plane VCN 718 can be integrated with customer tenancies 770. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 746. Code to run the function may be executed in the VMs 766(1)-(N), and the code may not be configured to run anywhere else on the data plane VCN 718. Each VM 766(1)-(N) may be connected to one customer tenancy 770. Respective containers 771(1)-(N) contained in the VMs 766(1)-(N) may be configured to run the code. In this case, there can be a dual isolation (e.g., the containers 771(1)-(N) running code, where the containers 771(1)-(N) may be contained in at least the VM 766(1)-(N) that are contained in the untrusted app subnet(s) 762), which may help prevent incorrect or otherwise undesirable code from damaging the network of the IaaS provider or from damaging a network of a different customer. The containers 771(1)-(N) may be communicatively coupled to the customer tenancy 770 and may be configured to transmit or receive data from the customer tenancy 770. The containers 771(1)-(N) may not be configured to transmit or receive data from any other entity in the data plane VCN 718. Upon completion of running the code, the IaaS provider may kill or otherwise dispose of the containers 771(1)-(N).

In some embodiments, the trusted app subnet(s) 760 may run code that may be owned or operated by the IaaS provider. In this embodiment, the trusted app subnet(s) 760 may be communicatively coupled to the DB subnet(s) 730 and be configured to execute CRUD operations in the DB subnet(s) 730. The untrusted app subnet(s) 762 may be communicatively coupled to the DB subnet(s) 730, but in this embodiment, the untrusted app subnet(s) may be configured to execute read operations in the DB subnet(s) 730. The containers 771(1)-(N) that can be contained in the VM 766(1)-(N) of each customer and that may run code from the customer may not be communicatively coupled with the DB subnet(s) 730.

In other embodiments, the control plane VCN 716 and the data plane VCN 718 may not be directly communicatively coupled. In this embodiment, there may be no direct communication between the control plane VCN 716 and the data plane VCN 718. However, communication can occur indirectly through at least one method. An LPG 710 may be established by the IaaS provider that can facilitate communication between the control plane VCN 716 and the data plane VCN 718. In another example, the control plane VCN 716 or the data plane VCN 718 can make a call to cloud services 756 via the service gateway 736. For example, a call to cloud services 756 from the control plane VCN 716 can include a request for a service that can communicate with the data plane VCN 718.

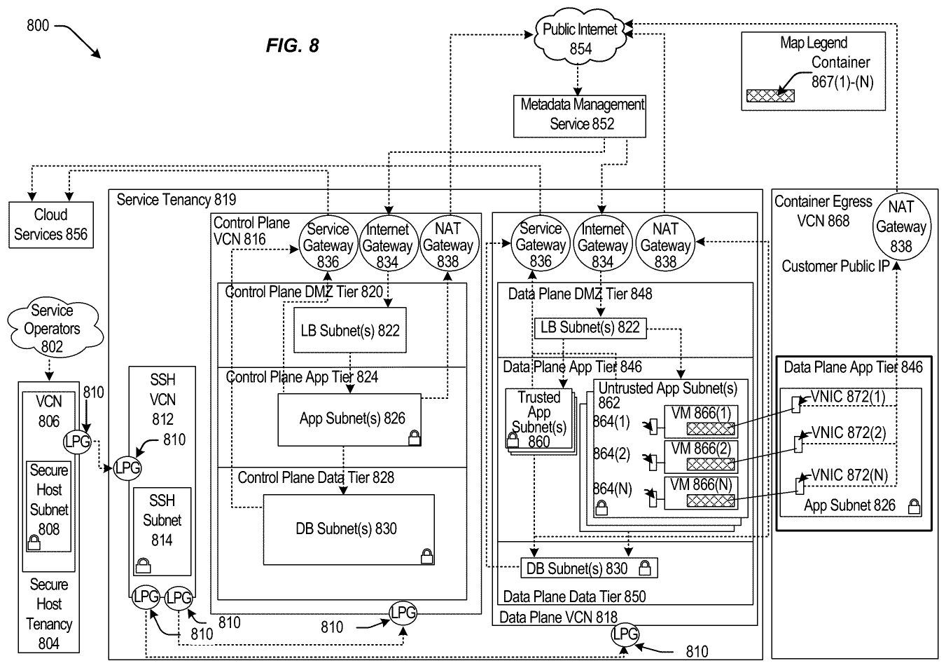

FIG. 8 is a block diagram 800 illustrating another example pattern of an IaaS architecture, according to at least one embodiment. Service operators 802 (e.g., service operators 502 of FIG. 5) can be communicatively coupled to a secure host tenancy 804 (e.g., the secure host tenancy 504 of FIG. 5) that can include a virtual cloud network (VCN) 806 (e.g., the VCN 506 of FIG. 5) and a secure host subnet 808 (e.g., the secure host subnet 508 of FIG. 5). The VCN 806 can include an LPG 810 (e.g., the LPG 510 of FIG. 5) that can be communicatively coupled to an SSH VCN 812 (e.g., the SSH VCN 512 of FIG. 5) via an LPG 810 contained in the SSH VCN 812. The SSH VCN 812 can include an SSH subnet 814 (e.g., the SSH subnet 514 of FIG. 5), and the SSH VCN 812 can be communicatively coupled to a control plane VCN 816 (e.g., the control plane VCN 516 of FIG. 5) via an LPG 810 contained in the control plane VCN 816 and to a data plane VCN 818 (e.g., the data plane 518 of FIG. 5) via an LPG 810 contained in the data plane VCN 818. The control plane VCN 816 and the data plane VCN 818 can be contained in a service tenancy 819 (e.g., the service tenancy 519 of FIG. 5).

The control plane VCN 816 can include a control plane DMZ tier 820 (e.g., the control plane DMZ tier 520 of FIG. 5) that can include LB subnet(s) 822 (e.g., LB subnet(s) 522 of FIG. 5), a control plane app tier 824 (e.g., the control plane app tier 524 of FIG. 5) that can include app subnet(s) 826 (e.g., app subnet(s) 526 of FIG. 5), a control plane data tier 828 (e.g., the control plane data tier 528 of FIG. 5) that can include DB subnet(s) 830 (e.g., DB subnet(s) 730 of FIG. 7). The LB subnet(s) 822 contained in the control plane DMZ tier 820 can be communicatively coupled to the app subnet(s) 826 contained in the control plane app tier 824 and to an Internet gateway 834 (e.g., the Internet gateway 534 of FIG. 5) that can be contained in the control plane VCN 816, and the app subnet(s) 826 can be communicatively coupled to the DB subnet(s) 830 contained in the control plane data tier 828 and to a service gateway 836 (e.g., the service gateway of FIG. 5) and a network address translation (NAT) gateway 838 (e.g., the NAT gateway 538 of FIG. 5). The control plane VCN 816 can include the service gateway 836 and the NAT gateway 838.

The data plane VCN 818 can include a data plane app tier 846 (e.g., the data plane app tier 546 of FIG. 5), a data plane DMZ tier 848 (e.g., the data plane DMZ tier 548 of FIG. 5), and a data plane data tier 850 (e.g., the data plane data tier 550 of FIG. 5). The data plane DMZ tier 848 can include LB subnet(s) 822 that can be communicatively coupled to trusted app subnet(s) 860 (e.g., trusted app subnet(s) 760 of FIG. 7) and untrusted app subnet(s) 862 (e.g., untrusted app subnet(s) 762 of FIG. 7) of the data plane app tier 846 and the Internet gateway 834 contained in the data plane VCN 818. The trusted app subnet(s) 860 can be communicatively coupled to the service gateway 836 contained in the data plane VCN 818, the NAT gateway 838 contained in the data plane VCN 818, and DB subnet(s) 830 contained in the data plane data tier 850. The untrusted app subnet(s) 862 can be communicatively coupled to the service gateway 836 contained in the data plane VCN 818 and DB subnet(s) 830 contained in the data plane data tier 850. The data plane data tier 850 can include DB subnet(s) 830 that can be communicatively coupled to the service gateway 836 contained in the data plane VCN 818.

The untrusted app subnet(s) 862 can include primary VNICs 864(1)-(N) that can be communicatively coupled to tenant virtual machines (VMs) 866(1)-(N) residing within the untrusted app subnet(s) 862. Each tenant VM 866(1)-(N) can run code in a respective container 867(1)-(N), and be communicatively coupled to an app subnet 826 that can be contained in a data plane app tier 846 that can be contained in a container egress VCN 868. Respective secondary VNICs 872(1)-(N) can facilitate communication between the untrusted app subnet(s) 862 contained in the data plane VCN 818 and the app subnet contained in the container egress VCN 868. The container egress VCN can include a NAT gateway 838 that can be communicatively coupled to public Internet 854 (e.g., public Internet 554 of FIG. 5).

The Internet gateway 834 contained in the control plane VCN 816 and contained in the data plane VCN 818 can be communicatively coupled to a metadata management service 852 (e.g., the metadata management system 552 of FIG. 5) that can be communicatively coupled to public Internet 854. Public Internet 854 can be communicatively coupled to the NAT gateway 838 contained in the control plane VCN 816 and contained in the data plane VCN 818. The service gateway 836 contained in the control plane VCN 816 and contained in the data plane VCN 818 can be communicatively couple to cloud services 856.

In some examples, the pattern illustrated by the architecture of block diagram 800 of FIG. 8 may be considered an exception to the pattern illustrated by the architecture of block diagram 700 of FIG. 7 and may be desirable for a customer of the IaaS provider if the IaaS provider cannot directly communicate with the customer (e.g., a disconnected region). The respective containers 867(1)-(N) that are contained in the VMs 866(1)-(N) for each customer can be accessed in real-time by the customer. The containers 867(1)-(N) may be configured to make calls to respective secondary VNICs 872(1)-(N) contained in app subnet(s) 826 of the data plane app tier 846 that can be contained in the container egress VCN 868. The secondary VNICs 872(1)-(N) can transmit the calls to the NAT gateway 838 that may transmit the calls to public Internet 854. In this example, the containers 867(1)-(N) that can be accessed in real-time by the customer can be isolated from the control plane VCN 816 and can be isolated from other entities contained in the data plane VCN 818. The containers 867(1)-(N) may also be isolated from resources from other customers.

In other examples, the customer can use the containers 867(1)-(N) to call cloud services 856. In this example, the customer may run code in the containers 867(1)-(N) that requests a service from cloud services 856. The containers 867(1)-(N) can transmit this request to the secondary VNICs 872(1)-(N) that can transmit the request to the NAT gateway that can transmit the request to public Internet 854. Public Internet 854 can transmit the request to LB subnet(s) 822 contained in the control plane VCN 816 via the Internet gateway 834. In response to determining the request is valid, the LB subnet(s) can transmit the request to app subnet(s) 826 that can transmit the request to cloud services 856 via the service gateway 836.

It should be appreciated that IaaS architectures 500, 600, 700, 800 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

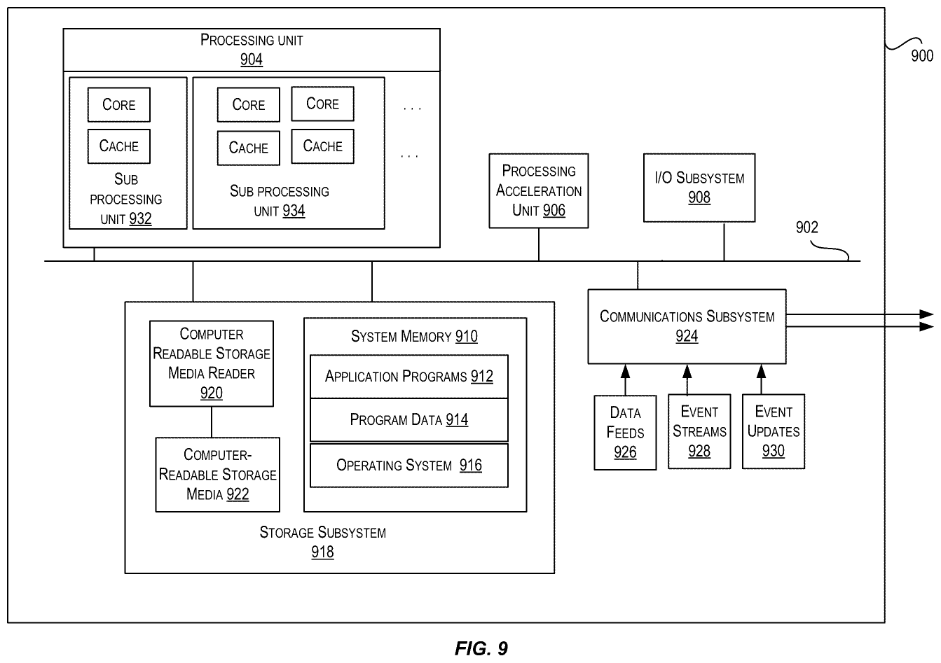

FIG. 9 illustrates an example computer system 900, in which various embodiments may be implemented. The system 900 may be used to implement any of the computer systems described above. As shown in the figure, computer system 900 includes a processing unit 904 that communicates with a number of peripheral subsystems via a bus subsystem 902. These peripheral subsystems may include a processing acceleration unit 906, an I/O subsystem 908, a storage subsystem 918 and a communications subsystem 924. Storage subsystem 918 includes tangible computer-readable storage media 922 and a system memory 910.

Bus subsystem 902 provides a mechanism for letting the various components and subsystems of computer system 900 communicate with each other as intended. Although bus subsystem 902 is shown schematically as a single bus, alternative embodiments of the bus subsystem may utilize multiple buses. Bus subsystem 902 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 904, which can be implemented as one or more integrated circuits (e.g., a conventional microprocessor or microcontroller), controls the operation of computer system 900. One or more processors may be included in processing unit 904. These processors may include single core or multicore processors. In certain embodiments, processing unit 904 may be implemented as one or more independent processing units 932 and/or 934 with single or multicore processors included in each processing unit. In other embodiments, processing unit 904 may also be implemented as a quad-core processing unit formed by integrating two dual-core processors into a single chip.

In various embodiments, processing unit 904 can execute a variety of programs in response to program code and can maintain multiple concurrently executing programs or processes. At any given time, some or all of the program code to be executed can be resident in processor(s) 904 and/or in storage subsystem 918. Through suitable programming, processor(s) 904 can provide various functionalities described above. Computer system 900 may additionally include a processing acceleration unit 906, which can include a digital signal processor (DSP), a special-purpose processor, and/or the like.

I/O subsystem 908 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 900 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 900 may comprise a storage subsystem 918 that comprises software elements, shown as being currently located within a system memory 910. System memory 910 may store program instructions that are loadable and executable on processing unit 904, as well as data generated during the execution of these programs.

Depending on the configuration and type of computer system 900, system memory 910 may be volatile (such as random access memory (RAM)) and/or non-volatile (such as read-only memory (ROM), flash memory, etc.) The RAM typically contains data and/or program modules that are immediately accessible to and/or presently being operated and executed by processing unit 904. In some implementations, system memory 910 may include multiple different types of memory, such as static random access memory (SRAM) or dynamic random access memory (DRAM). In some implementations, a basic input/output system (BIOS), containing the basic routines that help to transfer information between elements within computer system 900, such as during start-up, may typically be stored in the ROM. By way of example, and not limitation, system memory 910 also illustrates application programs 912, which may include client applications, Web browsers, mid-tier applications, relational database management systems (RDBMS), etc., program data 914, and an operating system 916. By way of example, operating system 916 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems.

Storage subsystem 918 may also provide a tangible computer-readable storage medium for storing the basic programming and data constructs that provide the functionality of some embodiments. Software (programs, code modules, instructions) that when executed by a processor provide the functionality described above may be stored in storage subsystem 918. These software modules or instructions may be executed by processing unit 904. Storage subsystem 918 may also provide a repository for storing data used in accordance with the present disclosure.

Storage subsystem 900 may also include a computer-readable storage media reader 920 that can further be connected to computer-readable storage media 922. Together and, optionally, in combination with system memory 910, computer-readable storage media 922 may comprehensively represent remote, local, fixed, and/or removable storage devices plus storage media for temporarily and/or more permanently containing, storing, transmitting, and retrieving computer-readable information.

Computer-readable storage media 922 containing code, or portions of code, can also include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media. This can also include nontangible computer-readable media, such as data signals, data transmissions, or any other medium which can be used to transmit the desired information and which can be accessed by computing system 900.

By way of example, computer-readable storage media 922 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 922 may include, but is not limited to, Zip® drives, flash memory cards, universal serial bus (USB) flash drives, secure digital (SD) cards, DVD disks, digital video tape, and the like. Computer-readable storage media 922 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 900.

Communications subsystem 924 provides an interface to other computer systems and networks. Communications subsystem 924 serves as an interface for receiving data from and transmitting data to other systems from computer system 900. For example, communications subsystem 924 may enable computer system 900 to connect to one or more devices via the Internet. In some embodiments communications subsystem 924 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof), global positioning system (GPS) receiver components, and/or other components. In some embodiments communications subsystem 924 can provide wired network connectivity (e.g., Ethernet) in addition to or instead of a wireless interface.

In some embodiments, communications subsystem 924 may also receive input communication in the form of structured and/or unstructured data feeds 926, event streams 928, event updates 930, and the like on behalf of one or more users who may use computer system 900.

By way of example, communications subsystem 924 may be configured to receive data feeds 926 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 924 may also be configured to receive data in the form of continuous data streams, which may include event streams 928 of real-time events and/or event updates 930, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 924 may also be configured to output the structured and/or unstructured data feeds 926, event streams 928, event updates 930, and the like to one or more databases that may be in communication with one or more streaming data source computers coupled to computer system 900.

Computer system 900 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 900 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or modules are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A method comprising:
accessing, by a summary generation system, reference information to be used for generating a summary for content to be summarized, wherein the reference information includes a set of original questions and a set of variant questions, the set of variant questions comprising multiple variant questions for each original question in the set of original questions;
creating, by the summary generation system, a plurality of chunks based upon the content to be summarized;
generating, by the summary generation system, an answer for each variant question in the set of variant questions, wherein an answer for a variant question is generated based upon one or more chunks from the plurality of chunks;
generating, by the summary generation system, an answer for each original question in the set of original questions based upon the answers generated for the one or more variant questions corresponding to the set of original questions;
selecting, by the summary generation system, a set of answers from the answers generated for the set of original questions; and
generating, by the summary generation system, using a first Machine Learning (ML) model and the selected set of answers for the set of original questions, a summary for the content to be summarized.

2. The method of claim 1, wherein generating, by the summary generation system, the summary for the content to be summarized using the first ML model comprises:
collating, by the summary generation system, the selected set of answers to form a collated result;
providing, by the summary generation system, the collated result as an input to the first ML model; and
responsive to the providing, generating, by the summary generation system, using the first ML model and the collated result, the summary for the content to be summarized.

3. The method of claim 1, wherein generating the answer for each variant question in the set of variant questions comprises:
using a first technique to identify, for each variant question in the set of variant questions and from the plurality of chunks, a first subset of chunks that are relevant to the variant question;
using a second technique to identify, for each variant question in the set of variant questions, a second subset of chunks for the variant question from the first subset of chunks identified for the variant question; and
for each variant question in the set of variant questions, generating an answer for the variant question based upon the second subset of chunks identified for the variant question and using a second machine learning (ML) model.

4. The method of claim 3, wherein using the first technique to identify, for each variant question in the set of variant questions and from the plurality of chunks, a first subset of chunks that are relevant to the variant question comprises:
generating a plurality of summaries for the plurality of chunks using a third machine learning (ML) model, wherein generating the plurality of summaries comprises generating a summary for each chunk in the plurality of chunks;
generating a first plurality of embeddings for the plurality of chunks;
generating a second plurality of embeddings for the plurality of summaries generated for the plurality of chunks; and
using the first plurality of embeddings, the second plurality of embeddings and an inverted search index created for the content to be summarized to rank the plurality of chunks based on relevance of each chunk in the plurality of chunks to the variant question.

5. The method of claim 4, wherein the inverted search index is created using a Best Match 25 (BM25) search function.

6. The method of claim 3, further comprising selecting the first subset of chunks for the variant question based on ranking the plurality of chunks based on relevance of each chunk in the plurality of chunks to the variant question.

7. The method of claim 3, wherein using the second technique to identify, for each variant question in the set of variant questions, a second subset of chunks for the variant question from the first subset of chunks identified for the variant question comprises:
for each variant question in the set of variant questions, using a cross encoder model to identify the second subset of chunks for the variant question from the first subset of chunks identified for the variant question, wherein a number of chunks in the second subset of chunks is less than a number of chunks in the first subset of chunks.

8. The method of claim 3, wherein generating an answer for the variant question based upon the second subset of chunks identified for the variant question comprises:
for each variant question in the set of variant questions, concatenating the chunks in the second subset of chunks to generate contextual content for the variant question; and
for each variant question in the set of variant questions, using the contextual content and the variant question to make a call to the second ML model to cause the second ML model to generate the answer for the variant question based upon the contextual content.

9. The method of claim 1, wherein generating an answer for each original question in the set of original questions based upon the answers generated for the one or more variant questions corresponding to the original questions comprises:
for each original question in the set of original questions, combining the answers generated for the one more variant questions corresponding to the original question to generate the answer for the original question.

10. The method of claim 1, wherein selecting a set of answers from the answers generated for the set of original questions comprises filtering one or more answers from the answers generated for the set of original questions using filtering criteria.

11. The method of claim 1, further comprising providing the summary as the summary for the content to be summarized.

12. A summary generation system comprising:
a memory; and
one or more processors configured to perform processing, the processing comprising:
accessing reference information to be used for generating a summary for content to be summarized, wherein the reference information includes a set of original questions and a set of variant questions, the set of variant questions comprising multiple variant questions for each original question in the set of original questions;
creating a plurality of chunks based upon the content to be summarized;
generating an answer for each variant question in the set of variant questions, wherein an answer for a variant question is generated based upon one or more chunks from the plurality of chunks;
generating an answer for each original question in the set of original questions based upon the answers generated for the one or more variant questions corresponding to the set of original questions;
selecting a set of answers from the answers generated for the set of original questions; and
generating using a first Machine Language (ML) model and the selected set of answers for the set of original questions, a summary for the content to be summarized.

13. The system of claim 12, wherein generating the answer for each variant question in the set of variant questions comprises:
using a first technique to identify, for each variant question in the set of variant questions and from the plurality of chunks, a first subset of chunks that are relevant to the variant question;
using a second technique to identify, for each variant question in the set of variant questions, a second subset of chunks for the variant question from the first subset of chunks identified for the variant question; and
for each variant question in the set of variant questions, generating an answer for the variant question based upon the second subset of chunks identified for the variant question and using a second machine learning (ML) model.

14. The system of claim 13, wherein using the first technique to identify, for each variant question in the set of variant questions and from the plurality of chunks, a first subset of chunks that are relevant to the variant question comprises:
for each variant question, ranking the plurality of chunks based on relevance of each chunk in the plurality of chunks to the variant question; and
for each variant question, selecting the first subset of chunks for the variant question based on ranking the plurality of chunks based on relevance of each chunk in the plurality of chunks to the variant question.

15. The system of claim 13, wherein using the second technique to identify, for each variant question in the set of variant questions, a second subset of chunks for the variant question from the first subset of chunks identified for the variant question comprises:
for each variant question in the set of variant questions, using a cross encoder model to identify the second subset of chunks for the variant question from the first subset of chunks identified for the variant question, wherein a number of chunks in the second subset of chunks is less than a number of chunks in the first subset of chunks.

16. The system of claim 12, wherein generating an answer for each original question in the set of original questions based upon the answers generated for the one or more variant questions corresponding to the original questions comprises:
for each original question in the set of original questions, combining the answers generated for the one more variant questions corresponding to the original question to generate the answer for the original question.

17. The system of claim 12, wherein selecting a set of answers from the answers generated for the set of original questions comprises filtering one or more answers from the answers generated for the set of original questions using filtering criteria.

18. One or more non-transitory computer-readable media storing instructions executable by a computer system that, when executed by one or more processors of the computer system, cause the computer system to perform operations comprising:
accessing reference information to be used for generating a summary for content to be summarized, wherein the reference information includes a set of original questions and a set of variant questions, the set of variant questions comprising multiple variant questions for each original question in the set of original questions;
creating a plurality of chunks based upon the content to be summarized;
generating an answer for each variant question in the set of variant questions, wherein an answer for a variant question is generated based upon one or more chunks from the plurality of chunks;
generating an answer for each original question in the set of original questions based upon the answers generated for the one or more variant questions corresponding to the set of original questions;
selecting a set of answers from the answers generated for the set of original questions; and
generating using a first Machine Language (ML) model and the selected set of answers for the set of original questions, a summary for the content to be summarized.

19. The non-transitory computer-readable medium of claim 18 wherein generating the summary for the content to be summarized using the first ML model comprises:
collating the selected set of answers to form a collated result;
providing collated result as an input to the first ML model; and
responsive to the providing, generating, using the first ML model and the collated result, the summary for the content to be summarized.

20. The non-transitory computer-readable medium of claim 18 wherein generating the answer for each variant question in the set of variant questions comprises:
using a first technique to identify, for each variant question in the set of variant questions and from the plurality of chunks, a first subset of chunks that are relevant to the variant question;
using a second technique to identify, for each variant question in the set of variant questions, a second subset of chunks for the variant question from the first subset of chunks identified for the variant question; and
for each variant question in the set of variant questions, generating an answer for the variant question based upon the second subset of chunks identified for the variant question and using a second machine learning (ML) model.
